(19) United States
(12) Patent Application Publication
(10) Pub. No.: US 2024/0109832 A1
(43) Pub. Date: Apr. 4, 2024

(54) RAPID CONSTRUCTION OF TETRALIN, CHROMANE, AND INDANE MOTIFS VIA CYCLATIVE C-H/C-H COUPLING: FOUR-STEP TOTAL SYNTHESIS OF (±)-RUSSUJAPONOL F

(71) Applicant: The Scripps Research Institute, La Jolla, CA (US)

(72) Inventors: Jin-Quan YU, San Diego, CA (US); Zhe ZHUANG, San Diego, CA (US)

(21) Appl. No.: 18/252,655

(22) PCT Filed: Nov. 9, 2021
(86) PCT No.: PCT/US21/58617

§ 371 Date: May 11, 2023

Related U.S. Application Data
(60) Provisional application No. 63/112,464, filed on Nov. 11, 2020.

(51) Int. Cl. C07C 51/353; C07C 29/149; C07C 67/08; C07D 311/58

(52) U.S. Cl. CPC C07C 51/353; C07C 29/149; C07C 67/08; C07D 311/58; C07C 2602/08; C07C 2602/10; C07C 2603/26

(57) ABSTRACT

Disclosed herein is a process for achieving a palladium-catalyzed cyclative C(sp3)-H/C(sp2)-H coupling reaction using a native free carboxylic acid as a directing group, an amino acid ligand, and oxidant. The process is useful for synthesizing a range of biologically important scaffolds, including tetralins, chromanes, and indanes.

### CLAIM OF PRIORITY

The present application claims the benefit of priority to U.S. Provisional Application No. 63/112,464 filed on Nov. 11, 2020, which application is incorporated herein as if fully set forth.

### STATEMENT OF GOVERNMENT SUPPORT

This invention was made with government support under grant number 2R01GM084019 awarded by the National Institutes of Health, and grant number CHE-1700982 awarded by the National Science Foundation. The government has certain rights in the invention.

### BACKGROUND

Carbon-carbon (C—C) bond formation constitutes one of the most important classes of reactions in organic synthesis. Because such bond formation has the potential to shorten synthesis, the past two decades have witnessed rapid developments in using C—H activation strategies for the construction of C—C bonds.¹ While most coupling methods require pre-functionalized coupling partners (e.g. organoborons and organohalides), C—H/C—H coupling reactions offer a complementary strategy to construct a C—C bond directly from two simple C—H bonds.² Compared to traditional coupling methods, this green and atom-economical approach is highly attractive because water is potentially the sole stoichiometric byproduct of this process. Previous reports focused on the coupling of two relatively reactive C(sp²)-H bonds for biaryl synthesis,³ whereas only a few reactions have been reported for the construction of more challenging C(sp³)-C(sp²) bonds. Because these existing reaction protocols require exogenous directing groups (DGs) to promote cyclometallation, additional steps to install and remove the DG are necessary.^{5,6} Additionally, reported methods pose practical limitations, such as the stoichiometric use of precious silver salts^{4b,c,5,6b,c} and harsh conditions^{4b,c,5a,b,6}—with temperatures as high as 160° C. being reported. Moreover, current methods for C(sp³)-H/C(sp²)-H coupling initiated by C(sp³)-H activation are largely limited to more reactive heterocyclic C(sp²)-H bonds.^{5a,b,6} Hence, the development of C(sp³)-H/C(sp²)-H coupling reactions that use both a practical oxidant and native substrates remains a significant challenge.

Recent advances in C—H functionalization have provided chemists with creative and strategic retrosynthetic disconnections that are otherwise difficult to achieve using traditional methods.⁷ However, for C—H functionalization strategies to truly improve the overall efficiency of synthesis, three criteria should be met: (1) the ability to use a wide range of simple starting materials to enable the synthesis of diverse natural product families; (2) the use of native functionalities as the DG; (3) the site-selectivity of C—H functionalization reactions should be precisely controllable. Yet, approaches that could meet the aforementioned criteria are challenging and uncommon.^{7a,8}

### SUMMARY

The present disclosure overcomes these challenges and others by providing, in various embodiments, a process for making a compound of formula (2):

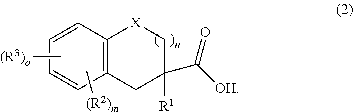

The process comprises contacting a compound of formula (1):

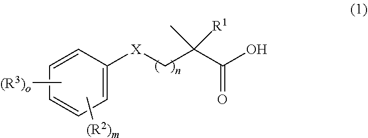

with a ligand of formula (L):

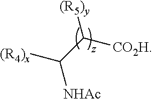

The contacting occurs in the presence of a source of palladium (II) and an oxidant, whereby a compound of formula (2) is formed.

In the process described herein:
X is CH₂ or O;
n is an integer selected from 0 and 1;
o and m are integers independently selected from 0, 1, and 2, wherein the sum of o and m is not greater than 4;
x and y are integers independently selected from 0 and 1;
z is an integer selected from 0, 1, and 2;
R¹ is selected from H and C₁-C₆-alkyl;
each R² and R³ is independently selected from the group consisting of C₁-C₆-alkyl, C₁-C₆-alkoxy, halo, C₁-C₆-haloalkyl, and (C₆-C₁₀-aryl)(C₁-C₆-alkyl)-;
or an adjacent R² and R³, together with the carbon atoms to which they are bound, form a fused C₅-C₆-cycloalkyl or phenyl; and
each R⁴ and R⁵ is independently selected from the group consisting of H, C₁-C₆-alkyl, and (C₆-C₁₀-aryl)(C₁-C₆-alkyl)-;
or, when z is 1, then R⁴ and R⁵ together with the carbon atoms to which they are bound form a 5- to 6-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl.

Additional embodiments of the disclosure are described in the accompanying drawings and detailed description.

### BRIEF DESCRIPTION OF THE DRAWINGS

FIG. 1. Biologically significant natural products contain tetralin, chromane, and indane frameworks.

FIG. 2. Ligand investigation in an exemplary cyclative C(sp³)-H/C(sp²)-H coupling reaction. Conditions: 1a (0.1 mmol), Pd(OAc)₂ (10 mol %), ligand (L) (10 mol %), LiOAc (1.0 equiv), Na₂CO₃·1.5H₂O₂ (2.0 equiv), HFIP (1.0 mL), 60° C., 12 h. The yields were determined by ¹H NMR analysis of the crude product using CH₂Br₂ as the internal standard. ^{c}Isolated yield.

FIG. 3. Substrate scope of an exemplary cyclative C(sp³)-H/C(sp²)-H coupling reaction with isolated yields. Conditions A: 1 (0.1 mmol), Pd(OAc)₂ (10 mol %), L9 (10 mol %), LiOAc (1.0 equiv), Na₂CO₃·1.5H₂O₂ (2.0 equiv), HFIP (1.0 mL), 60° C., 12 h. ^{c}Conditions B: 1 (0.1 mmol), Pd(CH₃CN)₄(BF₄)₂ (10 mol %), Ag₂CO₃ (1.0 equiv), 1-fluoro-2,4,6-trimethylpyridinium tetrafluoroborate (2.0 equiv), HFIP (1.0 mL), 90° C., 12 h.

FIG. 4. Illudalane sesquiterpenes have an indane core containing a quaternary center.

FIG. 5 Total synthesis of (±)-russujaponol F. Conditions: (a) SOCl₂, EtOH, reflux, overnight; I₂ (0.5 equiv), Selectfluor (0.5 equiv), CH₃CN, 60° C., 3 h. (b) Pd(OAc)₂ (10 mol %), L12 (10 mol %), pivalic acid (3.0 equiv), CsOAc (1.0 equiv), Ag₂CO₃ (2.0 equiv), HFIP, 80° C., 12 h. (c) Pd(CH₃CN)₄(BF₄)₂ (10 mol %), Ag₂CO₃ (1.0 equiv), 1-fluoro-2,4,6-trimethylpyridinium tetrafluoroborate (2.0 equiv), HFIP, 90° C., 12 h. (d) lithium aluminum hydride (LAH) (3.0 equiv), tetrahydrofuran (THF), 0° C. to rt, overnight.

### DETAILED DESCRIPTION

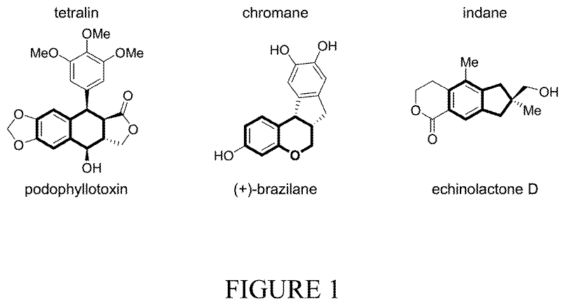

The present disclosure relates in part to a process for cyclative C(sp³)-H/C(sp²)-H coupling reaction using a native free carboxylic acid as the directing group (DG). In exemplary embodiments, a cyclopentane-based mono-N-protected β-amino acid ligand and a practical and inexpensive oxidant sodium percarbonate (Na₂CO₃·1.5H₂O₂) proved useful to the process. For instance, tetralins, chromanes, and indanes, which are common frameworks in natural products (FIG. 1), are readily prepared by this process. The synthetic application of this methodology is further demonstrated by a concise total synthesis of (±)-russujaponol F (the shortest and highest yielding to date) via multiple C—H functionalizations in four steps from readily available phenylacetic acid and pivalic acid (Scheme 1C), demonstrating the potential of C—H activation disconnections to enhance the ideality of synthesis⁹.

### Definitions

“Ac” refers to an acetyl group, having the formula —C(═O)—CH₃.

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl (“Me”), ethyl (“Et”), propyl, butyl (including t-butyl (“^{t}Bu”), pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups.

“Boc” refers to tert-Butyloxycarbonyl, having the formula (CH₃)₃C—O—C(═O)—

“Bn” refers to a benzyl group, having the formula —CH₂-phenyl.

Each of the terms “halogen,” “halide,” and “halo” refers to —F or fluoro, —Cl or chloro, —Br or bromo, or —I or iodo.

The term “alkoxy” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. In certain embodiments, the cycloalkyl group in the ligand of formula (L), in addition to having the —NHAc and the —CO₂H substituents as shown, is further substituted with 1-2 substituents selected from the group consisting of halo, C₁-C₆-alkyl, C₁-C₆-alkoxy, and C₆-C₁₀-aryl.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). An exemplary aryl is phenyl. An aryl group can be unsubstituted or optionally substituted with one or more substituents as described herein.

The term “optionally substituted” refers to optional substitution (i.e., unsubstituted or substituted) with the specified substituents.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In some embodiments of the process described herein, X is CH₂. In other embodiments, X is O.

In various embodiments, n is 0, while in other embodiments n is 1. In illustrative embodiments the compound of formula (2) is one selected from Table 1:

TABLE 1
Exemplary Compounds of formula (2).

| | |
|---|---|
| 2a | 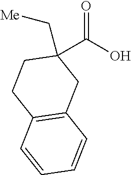 [SMILES: CCC1(C(=O)O)CCc2ccccc2C1] |
| 2b | 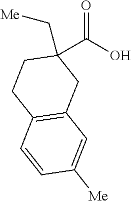 [SMILES: CCC1(C(=O)O)CCc2ccc(C)cc2C1] |
| 2c | 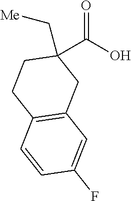 [SMILES: CCC1(C(=O)O)CCc2ccc(F)cc2C1] |
| 2d | 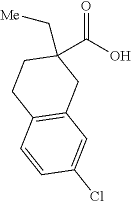 [SMILES: CCC1(C(=O)O)CCc2ccc(Cl)cc2C1] |
| 2e | 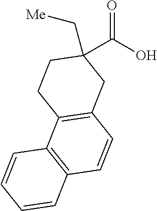 [SMILES: CCC1(C(=O)O)CCc2c(ccc3ccccc23)C1] |
| 2f | 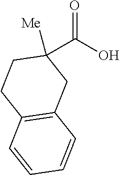 [SMILES: CC1(C(=O)O)CCc2ccccc2C1] |
| 2g | 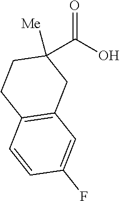 [SMILES: CC1(C(=O)O)CCc2ccc(F)cc2C1] |
| 2h | 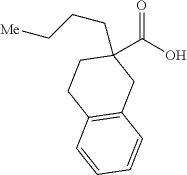 [SMILES: CCCCC1(C(=O)O)CCc2ccccc2C1] |
| 2i | 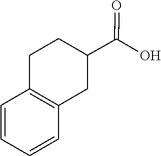 [SMILES: O=C(O)C1CCc2ccccc2C1] |
| 2j | 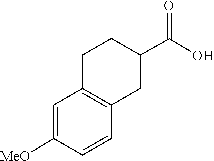 [SMILES: COc1ccc2c(c1)CCC(C(=O)O)C2] |
| 2k | 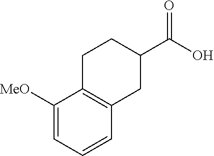 [SMILES: COc1cccc2c1CCC(C(=O)O)C2] |
| 2l | 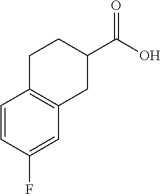 [SMILES: O=C(O)C1CCc2ccc(F)cc2C1] |
| 2m | 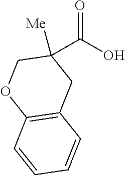 [SMILES: CC1(C(=O)O)COc2ccccc2C1] |
| 2n | 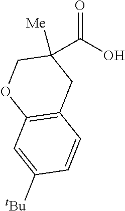 [SMILES: CC1(C(=O)O)COc2cc(C(C)(C)C)ccc2C1] |
| 2n′ | 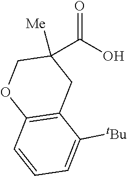 [SMILES: CC1(C(=O)O)COc2cccc(C(C)(C)C)c2C1] |
| 2o | 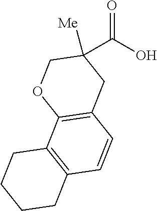 [SMILES: CC1(C(=O)O)COc2c(ccc3c2CCCC3)C1] |
| 2p | 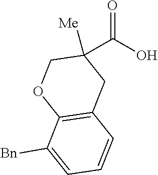 [SMILES: CC1(C(=O)O)COc2c(Cc3ccccc3)cccc2C1] |
| 2q | 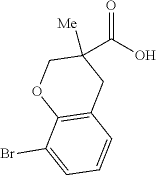 [SMILES: CC1(C(=O)O)COc2c(Br)cccc2C1] |
| 2r | 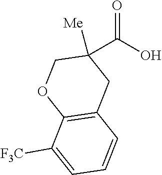 [SMILES: CC1(C(=O)O)COc2c(cccc2C(F)(F)F)C1] |
| 2s | 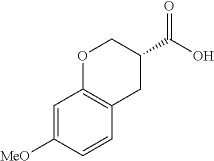 [SMILES: COc1ccc2c(c1)OC[C@H](C(=O)O)C2] |
| 2u | 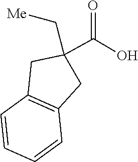 [SMILES: CCC1(C(=O)O)Cc2ccccc2C1] |
| 2v | 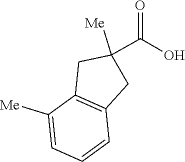 [SMILES: Cc1cccc2c1CC(C)(C(=O)O)C2] |
| 2w | 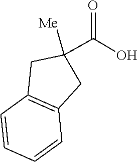 [SMILES: CC1(C(=O)O)Cc2ccccc2C1] |

In the ligand of formula (L), per various embodiments, z is 1. In other embodiments, z is 0 or 2.

In various embodiments, one of x and y is 0 and the other is 1. In additional embodiments, R⁴ and R⁵ together with the carbon atoms to which they are bound form a 5- to 6-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl. For example, per an embodiment, R⁴ and R⁵ together with the carbon atoms to which they are bound form a 5-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl. Per another embodiment, R⁴ and R⁵ together with the carbon atoms to which they are bound form a 6-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl. An exemplary ligand of formula (L) is one selected from Table 2.

TABLE 2
Exemplary ligands of formula (L).

| | |
|---|---|
| L1 | 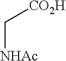 |
| L2 | 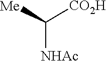 |
| L3 | 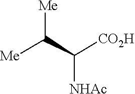 [SMILES: CC(=O)N[C@H](C(=O)O)C(C)C] |
| L4 | 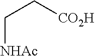 |
| L5 | 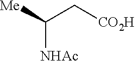 |
| L6 | 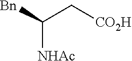 [SMILES: CC(=O)N[C@H](CC(=O)O)Cc1ccccc1] |
| L7 | 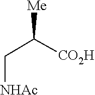 |
| L8 | 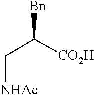 [SMILES: CC(=O)NC[C@@H](Cc1ccccc1)C(=O)O] |
| L9 | 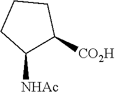 [SMILES: CC(=O)N[C@H]1CCC[C@H]1C(=O)O] |
| L10 | 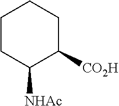 [SMILES: CC(=O)N[C@H]1CCCC[C@H]1C(=O)O] |
| L11 | 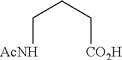 |

For instance, according to one embodiment, a useful ligand of formula (L) is L9:

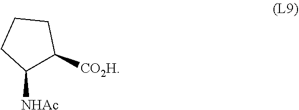 [SMILES: CC(=O)N[C@H]1CCC[C@H]1C(=O)O]

In various embodiments, the ligand of formula (L) is present in an amount of about 1 to about 15 mol % based upon the amount of compound of formula (2). For example, the amount can range from about 7 to about 12 mol %. The amount of ligand (L) in various embodiments is about 1, 2, 3, 4, 5, 6, 7, 8, 9, 10, 11, 12, 13, 14, or 15 mol %. In an illustrative embodiment, the amount is about 10 mol %.

In various embodiments, the compound of formula (1) is one chosen from Table 3.

TABLE 3
Exemplary compounds of formula (1).

| | |
|---|---|
| 1a | 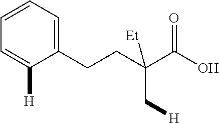 |
| 1b | 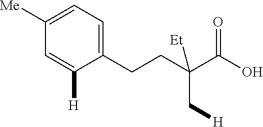 |
| 1c | 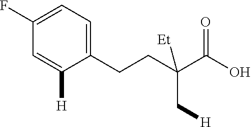 |
| 1d | 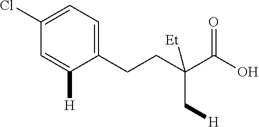 |
| 1e | 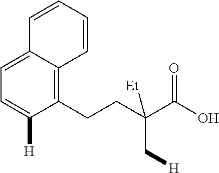 |
| 1f | 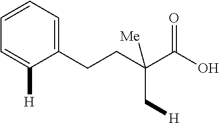 |
| 1g | 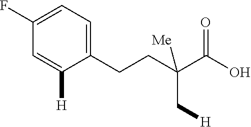 |
| 1h | 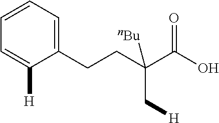 |
| 1i | 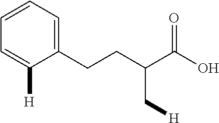 |
| 1j | 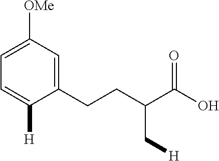 |
| 1k | 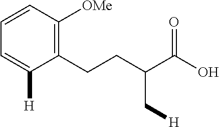 |
| 1l | 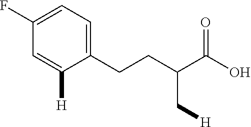 |
| 1m | 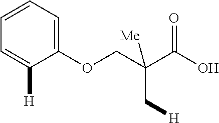 |
| 1n | 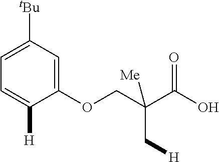 |
| 1o | 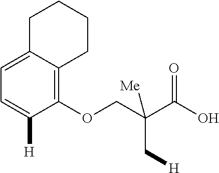 |
| 1p | 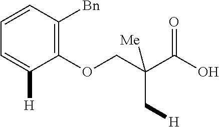 |
| 1q | 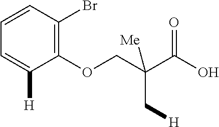 |
| 1r | 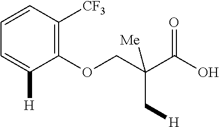 |
| 1s | 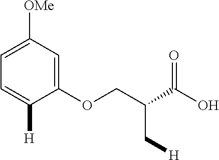 |
| 1t | 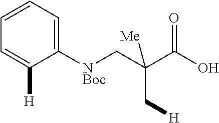 |
| 1u | 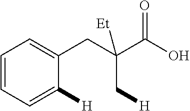 |
| 1v | 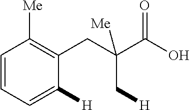 |
| 1w | 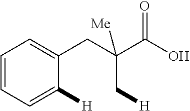 |

In the process described herein, a palladium catalyst arises from the introduction of palladium (II) via reagents known in the art or commercially available. One convenient source of palladium (II), per an embodiment, is Pd(OAc)₂. In another embodiment, the source is Pd(CH₃CN)₄(BF₄)₂.

Catalyst loading can vary in accordance with factors known to those skilled in the art, such as overall reaction kinetics. Thus, in various embodiments, the source of palladium (II) is present in amount of about 1 to about 15 mol % based upon the amount of compound of formula (2). In other embodiments, the amount is from about 7 to about 12 mol %. Exemplary amounts include 1, 2, 3, 4, 5, 6, 7, 8, 9, 10, 11, 12, 13, 14, and 15 mol %. In an embodiment, the amount is 10 mol %.

Various oxidants known in the art are useful in the process of the present disclosure. As described in more detail herein, according to an embodiment, a convenient oxidant is sodium percarbonate.

In various embodiments, the contacting step of the process described herein occurs further in presence of LiOAc. A useful solvent among others, in an embodiment, is hexafluoroisopropanol.

The process described herein can be carried out at various temperatures, in accordance with embodiments of the disclosure. For example, the temperature is about 20, 25, 30, 35, 40, 45, 50, 55, 60, 65, 70, 75, or 80° C. In an illustrative embodiment, the temperature is about 60° C.

In various embodiments, the ligand of formula (L) is (L9) present in an amount of about 10 mol %:

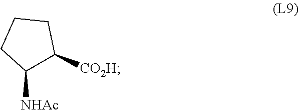 [SMILES: CC(=O)N[C@H]1CCC[C@H]1C(=O)O]

the sum of o and m is 1 or 2; the source of palladium (II) is Pd(OAc)₂ in amount of about 10 mol %; and the oxidant is sodium percarbonate.

Aliphatic carboxylic acids are ubiquitous and synthetically versatile motifs and are often inexpensive reagents in organic chemistry; as such, they are privileged substrates for C—H activation reactions.¹⁰ Following recent disclosure of the β-C(sp³)-H lactonization¹⁰ⁱ and acyloxylation^{10j} of free carboxylic acids using tert-butyl hydrogen peroxide (TBHP) as the sole oxidant, we initiated our investigation of cyclative C(sp³)-H/C(sp²)-H coupling reactions by selecting TBHP as the bystanding oxidant and aliphatic acid 1a as a model substrate, in accordance with an embodiment of the present disclosure. Under the optimal conditions of the aforementioned β-acyloxylation reaction^{10j}, a 50% ¹H NMR yield of the desired product 2a resulted without forming competing reductive elimination products, such as the β-lactone or β-hydroxy acid. Further investigation of the bystanding oxidants and bases revealed that, in various embodiments, a combination of Na₂CO₃·1.5H₂O₂ and LiOAc improves the yield to 57% (see examples). In some embodiments, the use of sodium percarbonate, one of the cheapest and most easily handled oxidants,¹¹ is one advantage rendering the process practical and scalable.

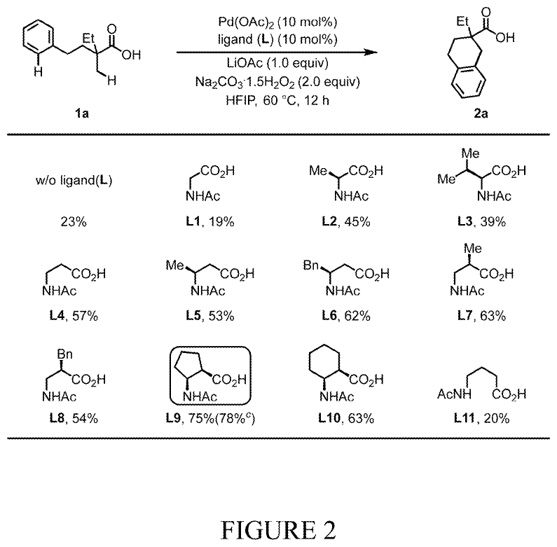

In light of recent advances in ligand-accelerated Pd(II)-catalyzed C—H activation,¹² we next searched for ligands that could substantially improve the reactivity of the catalyst. Guided by mono-N-protected amino acid (MPAA) ligand-enabled C(sp³)-H activation reactions of free carboxylic acids^{10c,d,g,i,j}, we tested a series of commercially available MPAA ligands (L1-L4): β-amino acid ligand L4 showed superior reactivity over α-amino acid ligands L1-L3 (57% vs. 19-45%), as was also observed in other C(sp³)-H functionalization reactions of free acids via Pd(II)/Pd(IV) catalytic cycles^{10d,i,j}. Through systematic modifications to the backbone of the β-amino acid ligand (L5-L10; see FIG. 2), it was surprisingly discovered that, in one embodiment, cis-cyclopentane-based ligand L9 gave the optimal reactivity (78% isolated yield). Without being bound to any particular theory, the superior reactivity of L9 can be attributed to the more rigid conformation enforced by the cyclopentane linkage. Control experiments showed that the yields were low in the absence of the ligand or in the presence of the γ-amino acid ligand (L11) (23% or 20%, respectively).

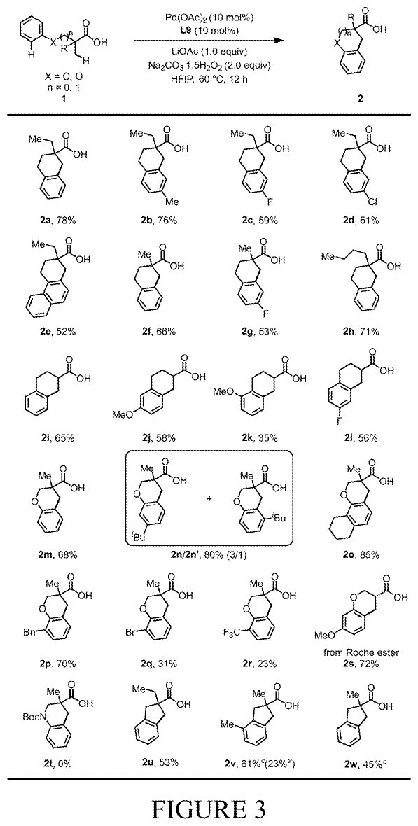

In addition, the scope of the cyclative C(sp³)-H/C(sp²)-H coupling reaction was evaluated through various embodiments (FIG. 3). A wide range of tertiary aliphatic acids bearing a single α-methyl group (1a-1e and 1h) or α-gem-dimethyl groups (1f and 1g) were all compatible, affording the tetralin products in moderate to good yields (52-78%). Less reactive free carboxylic acids containing α-hydrogens (1i-1l) also reacted in synthetically useful yields (35-65%). Among these, a variety of functionalities on the aryl rings such as methyl (2b), methoxy (2j and 2k), fluoro (2c, 2g, and 2l), and chloro (2d) as well as naphthyl (2e) were tolerated, with the halogen moiety (2d) serving as a useful synthetic handle for subsequent derivatization.

The process of the present disclosure is also useful in the synthesis of biologically important chromane products. For example, per various embodiments, β-Phenoxy carboxylic acids containing α-gem-dimethyl groups (1m-1r) or α-hydrogens (1s, from Roche ester) were all reactive substrates. While a range of electron-donating (methoxy, tert-butyl, cyclohexyl, and benzyl) (2s and 2n-2p) groups on the aryl ring were well tolerated to afford the desired products in good yields (70-85%), aliphatic acids containing electron-withdrawing (bromo and trifluoromethyl) groups (2q and 2r) showed comparatively low reactivity (31% and 23%), likely due to the sluggish nature of C(sp²)-H activations of electron-deficient arenes. Under the current conditions, carboxylic acid 1t failed to deliver tetrahydroisoquinoline (THIQ) product 2t. This cyclative C—H/C—H coupling reaction is also amenable to the syntheses of indane scaffolds (2u-2w). For example, in an embodiment, an [F⁺] oxidant^{3g,13} (1-fluoro-2,4,6-trimethylpyridinium tetrafluoroborate) showed superior reactivity for tertiary aliphatic acids containing α-gem-dimethyl groups (2v and 2w).

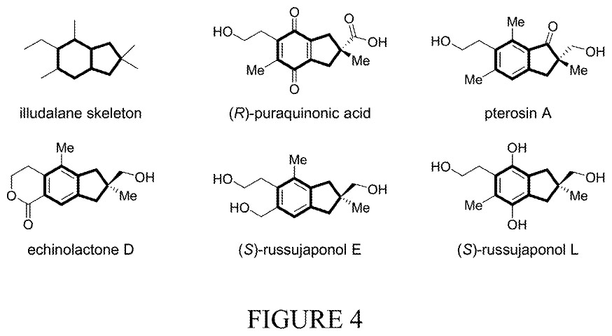

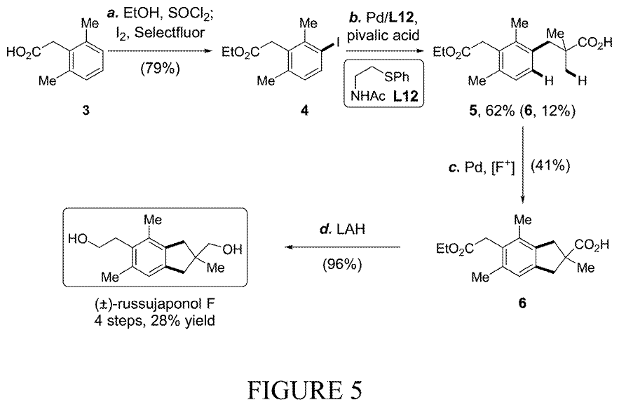

Additional embodiments illustrate the process of the present disclosure, concerning illudalane sesquiterpenes, which comprise a large family of natural products: these typically feature an indane core (for which various oxidation states are possible) bearing a challenging all-carbon quaternary center (FIG. 4).¹⁴ Owing to their promising biological activities, tremendous efforts have been devoted to the total syntheses of these targets.^{15,16} Given the power of this methodology for the construction of indane scaffolds, we embarked on the total synthesis of (±)-russujaponol F via multiple C—H functionalizations (FIG. 5). The first total synthesis of russujaponol F was reported to occur in racemic and enantioselective forms based on a C(sp³)-H arylation strategy in 13 steps (26% yield) and 15 steps (12% yield) respectively.¹⁵ Beginning with phenylacetic acid 3 that is commercially available or synthesized through ortho-C—H methylation¹⁷, we prepared aryl iodide 4 by esterification and subsequent mono-iodination¹⁸ of 3 using I₂ and Selectfluor in 79% yield. Investigation of the C—H arylation of pivalic acid indicated that, with ligand L12^{10f,19}, the mono-arylated product 5 could be obtained in 62% yield, along with 12% of the cyclative C—H/C—H coupling product 6. The formation of 6 under these conditions can be attributed to a second arylation of 5 with additional aryl iodide serving as the bystanding oxidant.²⁰ The cyclative C—H/C—H coupling was then performed under the standard conditions using an [F⁺] oxidant to give the desired product 6 in 41% yield. Finally, global reduction of 6 using LAH cleanly delivered (±)-russujaponol F in 96% yield, completing the total synthesis in four steps and 28% overall yield: the shortest and highest yielding total synthesis of russujaponol F to date.

Numbered references in the preceding sections are as follows:
(1) For reviews on C—H activation/C—C bond-forming reactions, see: (a) Chen, X.; Engle, K. M.; Wang, D.-H.; Yu, J.-Q. Palladium(II)-catalyzed C—H activation/C—C cross-coupling reactions: versatility and practicality. Angew. Chem., Int. Ed. 2009, 48, 5094-5115. (b) Daugulis, O.; Roane, J.; Tran, L. D. Bidentate, monoanionic auxiliary-directed functionalization of carbon-hydrogen bonds. Acc. Chem. Res. 2015, 48, 1053-1064. (c) He, G.; Wang, B.; Nack, W. A.; Chen, G. Syntheses and transformations of α-amino acids via palladium-catalyzed auxiliary directed sp³ C—H Functionalization. Acc. Chem. Res. 2016, 49, 635-645.
(2) For reviews on C—H/C—H coupling reactions, see: (a) Yeung, C. S.; Dong, V. M. Catalytic dehydrogenative cross-coupling: forming carbon-carbon bonds by oxidizing two carbon-hydrogen bonds. Chem. Rev. 2011, 111, 1215-1292. (b) Girard, S. A.; Knauber, T.; Li, C.-J. The cross-dehydrogenative coupling of C(sp³)-H bonds: a versatile strategy for C—C bond formations. Angew. Chem., Int. Ed. 2014, 53, 74-100. (c) Liu, C.; Yuan, J.; Gao, M.; Tang, S.; Li, W.; Shi, R.; Lei, A. Oxidative coupling between two hydrocarbons: an update of recent C—H functionalizations. Chem. Rev. 2015, 115, 12138-12204.
(3) For early examples of C(sp²)-H/C(sp²)-H coupling reaction, see: (a) Stuart, D. R.; Fagnou, K. The catalytic cross-coupling of unactivated arenes. Science 2007, 316, 1172-1175. (b) Xia, J.-B.; You, S.-L. Carbon-carbon bond formation through double sp² C—H activations: synthesis of ferrocenyl oxazoline derivatives. Organometallics 2007, 26, 4869-4871. (c) Hull, K. L.; Sanford, M. S. Catalytic and highly regioselective cross-coupling of aromatic C—H substrates. J. Am. Chem. Soc. 2007, 129, 11904-11905. (d) Brasche, G.; Garcia-Fortanet, J.; Buchwald, S. L. Twofold C—H functionalization: palladium-catalyzed ortho arylation of anilides. Org. Lett. 2008, 10, 2207-2210. (e) Cho, S. H.; Hwang, S. J.; Chang, S. Palladium-catalyzed C—H functionalization of pyridine N-oxides: highly selective alkenylation and direct arylation with unactivated arenes. J. Am. Chem. Soc. 2008, 130, 9254-9256. (f) Zhao, X.; Yeung, C. S.; Dong, V. M. Palladium-catalyzed ortho-arylation of O-phenylcarbamates with simple arenes and sodium persulfate. J. Am. Chem. Soc. 2010, 132, 5837-5844. (g) Wang, X.; Leow, D.; Yu, J.-Q. Pd(II)-catalyzed para-selective C—H arylation of monosubstituted arenes. J. Am. Chem. Soc. 2011, 133, 13864-13867.
(4) For Pd-catalyzed C(sp³)-H/C(sp²)-H coupling reactions initiated by C(sp²)-H activation, see: (a) Liegault, B.; Fagnou, K. Palladium-catalyzed intramolecular coupling of arenes and unactivated alkanes in air. Organometallics 2008, 27, 4841-4843. (b) Pierre, C.; Baudoin, O. Intramolecular Pd^{II}-catalyzed dehydrogenative C(sp³)-C(sp²) coupling: an alternative to Pd⁰-catalyzed C(sp³)-H arylation from aryl halides? Tetrahedron 2013, 69, 4473-4478. (c) Shi, J.-L.; Wang, D.; Zhang, X.-S.; Li, X.-L.; Chen, Y.-Q.; Li, Y.-X.; Shi, Z.-J. Oxidative coupling of sp² and sp³ carbon-hydrogen bonds to construct dihydrobenzofurans. Nat. Commun. 2017, 8, 238-244.
(5) For Pd-catalyzed C(sp³)-H/C(sp²)-H coupling reactions initiated by C(sp³)-H activation, see: (a) Jiang, Y.; Deng, G.; Zhang, S.; Loh, T.-P. Directing group participated benzylic C(sp³)-H/C(sp²)-H cross-dehydrogenative coupling (CDC): synthesis of azapolycycles. Org. Lett. 2018, 20, 652-655. (b) Sun, W.-W.; Liu, J.-K.; Wu, B. Practical synthesis of polysubstituted unsymmetric 1,10-phenanthrolines by palladium catalyzed intramolecular oxidative cross coupling of C(sp²)-H and C(sp³)-H bonds of carboxamides. Org. Chem. Front. 2019, 6, 544-550. (c) Hao, H.-Y.; Mao, Y.-J.; Xu, Z.-Y.; Lou, S.-J.; Xu, D.-Q. Selective cross-dehydrogenative C(sp³)-H arylation with arenes. Org. Lett. 2020, 22, 2396-2402.
(6) For other metal-enabled C(sp³)-H/C(sp²)-H coupling reactions, see: (a) Wu, X.; Zhao, Y.; Ge, H. Pyridine-enabled copper-promoted cross dehydrogenative coupling of C(sp²)-H and unactivated C(sp³)-H bonds. Chem. Sci. 2015, 6, 5978-5983. (b) Tan, G.; You, J. Rhodium(III)-catalyzed oxidative cross-coupling of unreactive C(sp³)-H bonds with C(sp²)-H bonds. Org. Lett. 2017, 19, 4782-4785. (c) Wang, X.; Xie, P.; Qiu, R.; Zhu, L.; Liu, T.; Li, Y.; Iwasaki, T.; Au, C.-T.; Xu, X.; Xia, Y.; Yin, S.-F.; Kambe, N. Nickel-catalysed direct alkylation of thiophenes via double C(sp³)-H/C(sp²)-H bond cleavage: the importance of KH₂PO₄. Chem. Commun. 2017, 53, 8316-8319. (d) Tan, G.; Zhang, L.; Liao, X.; Shi, Y.; Wu, Y.; Yang, Y.; You, J. Copper- or nickel-enabled oxidative cross-coupling of unreactive C(sp³)-H bonds with azole C(sp²)-H bonds: rapid access to β-azolyl propanoic acid derivatives. Org. Lett. 2017, 19, 4830-4833.
(7) For reviews on C—H functionalization for natural product synthesis, see: (a) Baudoin, O. Multiple catalytic C—H bond functionalization for natural product synthesis. Angew. Chem., Int. Ed. 2020, 59, 17798-17809. (b) Lam, N. Y. S.; Wu, K.; Yu, J.-Q. Advancing the logic of chemical synthesis: C—H activation as strategic and tactical disconnections for C—C bond construction. Angew. Chem., Int. Ed. 2020, 59, DOI: 10.1002/anie.202011901. (c) Gutekunst, W. R.; Baran, P. S. C—H functionalization logic in total synthesis. Chem. Soc. Rev. 2011, 40, 1976-1991. (d) Abrams, D. J.; Provencher, P. A.; Sorensen, E. J. Recent applications of C—H functionalization in complex natural product synthesis. Chem. Soc. Rev. 2018, 47, 8925-8967.
(8) For selected examples of total synthesis using multiple C—H functionalizations, see: (a) Wang, D.-H.; Yu, J.-Q. Highly convergent total synthesis of (+)-lithospermic acid via a late-stage intermolecular C—H olefination. J. Am. Chem. Soc. 2011, 133, 5767-5769. (b) Gutekunst, W. R.; Baran, P. S. Total synthesis and structural revision of the piperarborenines via sequential cyclobutane C—H arylation. J. Am. Chem. Soc. 2011, 133, 19076-19079. (c) Rosen, B. R.; Simke, L. R.; Thuy-Boun, P. S.; Dixon, D. D.; Yu, J.-Q.; Baran, P. S. C—H functionalization logic enables synthesis of (+)-hongoquercin A and related compounds. Angew. Chem., Int. Ed. 2013, 52, 7317-7320. (d) Hong, B.; Li, C.; Wang, Z.; Chen, J.; Li, H.; Lei, X. Enantioselective total synthesis of (−)-incarviatone A. J. Am. Chem. Soc. 2015, 137, 11946-11949. (e) Dailler, D.; Danoun, G.; Ourri, B.; Baudoin, O. Divergent synthesis of aeruginosins based on a C(sp³)-H activation strategy. Chem. Eur. J. 2015, 21, 9370-9379. (f) Wu, F.; Zhang, J.; Song, F.; Wang, S.; Guo, H.; Wei, Q.; Dai, H.; Chen, X.; Xia, X.; Liu, X.; Zhang, L.; Yu, J.-Q.; Lei, X. Chrysomycin A derivatives for the treatment of multi-drug-resistant tuberculosis. ACS Cent. Sci. 2020, 6, 928-938.
(9) Gaich, T.; Baran, P. S. Aiming for the ideal synthesis. J. Org. Chem. 2010, 75, 4657-4673.
(10) For β-C(sp³)-H functionalization reactions of free carboxylic acids, see: (a) Giri, R.; Maugel, N.; Li, J.-J.; Wang, D.-H.; Breazzano, S. P.; Saunder, L. B.; Yu, J.-Q. Palladium-catalyzed methylation and arylation of sp² and sp³ C—H bonds in simple carboxylic acids. J. Am. Chem. Soc. 2007, 129, 3510-3511. (b) Chen, G.; Zhuang, Z.; Li, G.-C; Saint-Denis, T. G.; Hsiao, Y.; Joe, C. L.; Yu, J.-Q. Ligand-enabled β-C—H arylation of α-amino acids without installing exogenous directing groups. Angew. Chem., Int. Ed. 2017, 56, 1506-1509. (c) Zhu, Y.; Chen, X.; Yuan, C.; Li, G.; Zhang, J.; Zhao, Y. Pd-catalysed ligand-enabled carboxylate-directed highly regioselective arylation of aliphatic acids. Nat. Commun. 2017, 8, 14904. (d) Ghosh, K. K.; van Gemmeren, M. Pd-catalyzed β-C(sp³)-H arylation of propionic acid and related aliphatic acids. Chem. Eur. J. 2017, 23, 17697-17700. (e) Shen, P.-X.; Hu, L.; Shao, Q.; Hong, K.; Yu, J.-Q. Pd(II)-catalyzed enantioselective C(sp³)-H arylation of free carboxylic acids. J. Am. Chem. Soc. 2018, 140, 6545-6549. (f) Zhuang, Z.; Yu, C.-B.; Chen, G.; Wu, Q.-F.; Hsiao, Y.; Joe, C. L.; Qiao, J. X.; Poss, M. A.; Yu, J.-Q. Ligand-enabled β-C(sp³)-H olefination of free carboxylic acids. J. Am. Chem. Soc. 2018, 140, 10363-10367. (g) Hu, L.; Shen, P.-X.; Shao, Q.; Hong, K.; Qiao, J. X.; Yu, J.-Q. Pd^{II}-catalyzed enantioselective C(sp³)-H activation/cross-coupling reactions of free carboxylic acids. Angew. Chem., Int. Ed. 2019, 58, 2134-2138. (h) Ghosh, K. K.; Uttry, A.; Koldemir, A.; Ong, M.; van Gemmeren, M. Direct β-C(sp³)-H acetoxylation of aliphatic carboxylic acids. Org. Lett. 2019, 21, 7154-7157. (i) Zhuang, Z.; Yu, J.-Q. Lactonization as a general route to β-C(sp³)-H functionalization. Nature 2020, 577, 656-659. (j) Zhuang, Z.; Herron, A. N.; Fan, Z.; Yu, J.-Q. Ligand-enabled monoselective β-C(sp³)-H acyloxylation of free carboxylic acids using a practical oxidant. J. Am. Chem. Soc. 2020, 142, 6769-6776. (k) Ghiringhelli, F.; Uttry, A.; Ghosh, K. K.; van Gemmeren, M. Direct β- and γ-C(sp³)-H alkynylation of free carboxylic acids. Angew. Chem., Int. Ed. 2020, 59, DOI: 10.1002/anie.202010784.
(11) (a) McKillop, A.; Sanderson, W. R. Sodium perborate and sodium percarbonate: Cheap, safe and versatile oxidising agents for organic synthesis. Tetrahedron Lett. 1995, 51, 6145. (b) Muzart, J. Sodium perborate and sodium percarbonate in organic synthesis. Synthesis 1995, 1325.
(12) For reviews, see: (a) He, J.; Wasa, M.; Chan, K. S. L.; Shao, Q.; Yu, J.-Q. Palladium-catalyzed transformations of alkyl C—H bonds. Chem. Rev. 2017, 117, 8754-8786. (b) Shao, Q.; Wu, K.; Zhuang, Z.; Qian, S.; Yu, J.-Q. From Pd(OAc)₂ to chiral catalysts: the discovery and development of bifunctional mono-N-protected amino acid ligands for diverse C—H activation reactions. Acc. Chem. Res. 2020, 53, 833-851.
(13) Engle, K. M.; Mei, T.-S.; Wang, X.; Yu, J.-Q. Bystanding F⁺ oxidants enable selective reductive elimination from high-valent metal centers in catalysis. Angew. Chem., Int. Ed. 2011, 50, 1478-1491.
(14) (a) Yoshikawa, K.; Kaneko, A.; Matsumoto, Y.; Hama, H.; Arihara, S. Russujaponols A-F, illudoid sesquiterpenes from the fruiting body of Russula japonica. J. Nat. Prod. 2006, 69, 1267-1270. (b) Yoshikawa, K.; Matsumoto, Y.; Hama, H.; Tanaka, M.; Zhai, H.; Fukuyama, Y.; Arihara, S.; Hashimoto, T. Russujaponols G-L, illudoid sesquiterpenes, and their neurite outgrowth promoting activity from the fruit body of Russula japonica. Chem. Pharm. Bull. 2009, 57, 311-314. (c) Becker, U.; Erkel, G.; Anke, T.; Sterner, O. Puraquinonic acid, a novel inducer of differentiation of human HL-60 promyelocytic leukemia cells from Mycena puna (Pers. Ex Fr.). Nat. Prod. Lett. 1997, 9, 229-236. (d) Kuroyanagi, M.; Fukuoka, M.; Yoshihira, K.; Natori, S. The absolute configurations of pterosins, 1-indanone derivatives from bracken, Pteridium aquilinum var. latiusculum. Chem. Pharm. Bull. 1974, 22, 723-726. (e) Suzuki, S.; Murayama, T.; Shiono, Y. Echinolactones C and D: two illudalane sesquiterpenoids isolated from the cultured mycelia of the fungus Echinodontium japonicum. Z. Naturforsch., B 2006, 61, 1295-1298.
(15) (a) Melot, R.; Craveiro, M.; Btirgi, T.; Baudoin, O. Divergent enantioselective synthesis of (nor)illudalane sesquiterpenes via Pd⁰-catalyzed asymmetric C(sp³)-H activation. Org. Lett. 2019, 21, 812-815. (b) Melot, R.; Craveiro, M. V.; Baudoin, O. Total synthesis of (nor)illudalane sesquiterpenes based on a C(sp³)-H activation strategy. J. Org. Chem. 2019, 84, 12933-12945.
(16) For recent examples, see: (a) Tiong, E. A.; Rivalti, D.; Williams, B. M.; Gleason, J. L. A concise total synthesis of (R)-puraquinonic acid. Angew. Chem., Int. Ed. 2013, 52, 3442-3445. (b) Elmehriki, A. A. H.; Gleason, J. L. A spiroalkylation method for the stereoselective construction of a-quaternary carbons and its application to the total synthesis of (R)-puraquinonic acid. Org. Lett. 2019, 21, 9729-9733. (c) Zeng, Z.; Zhao, Y.; Zhang, Y. Divergent total syntheses of five illudalane sesquiterpenes and assignment of the absolute configuration. Chem. Commun. 2019, 55, 4250-4253. (d) Xun, M. M.; Bai, Y.; Wang, Y.; Hu, Z.; Fu, K.; Ma, W.; Yuan, C. Synthesis of four illudalane sesquiterpenes utilizing a one-pot Diels-Alder/oxidative aromatization sequence. Org. Lett. 2019, 21, 6879-6883.
(17) Thuy-Boun, P. S.; Villa, G.; Dang, D.; Richardson, P.; Su, S.; Yu, J.-Q. Ligand-accelerated ortho-C—H alkylation of arylcarboxylic acids using alkyl boron reagents. J. Am. Chem. Soc. 2013, 135, 17508-17513.
(18) Stavber, S.; Kralj, P.; Zupan, M. Selective and effective iodination of alkyl-substituted benzenes with elemental iodine activated by Selectfluor™ F-TEDA-BF₄. Synlett 2002, 598-600.
(19) For examples of C—H activation reactions using L12, see: (a) Le, K. K. A.; Nguyen, H.; Daugulis, O. 1-Aminopyridinium ylides as monodentate directing groups for sp³ C—H bond functionalization. J. Am. Chem. Soc. 2019, 141, 14728-14735. (b) Zhuang, Z.; Yu, J.-Q. Pd(II)-catalyzed enantioselective γ-C(sp³)-H functionalizations of free cyclopropylmethylamines. J. Am. Chem. Soc. 2020, 142, 12015-12019.
(20) (a) Sun, W.-W.; Cao, P.; Mei, R.-Q.; Li, Y.; Ma, Y.-L.; Wu, B. Palladium-catalyzed unactivated C(sp³)-H bond activation and intramolecular amination of carboxamides: a new approach to β-lactams. Org. Lett. 2014, 16, 480-483. (b) Zhang, S.-J.; Sun, W.-W.; Cao, P.; Dong, X.-P.; Liu, J.-K.; Wu, B. Stereoselective synthesis of diazabicyclic β-lactams through intramolecular amination of unactivated C(sp³)-H Bonds of carboxamides by palladium catalysis. J. Org. Chem. 2016, 81, 956-968. (c) Tong, H.-R.; Zheng, W.; Lv, X.; He, G.; Liu, P.; Chen, G. Asymmetric synthesis of β-lactam via palladium-catalyzed enantioselective intramolecular C(sp³)-H amidation. ACS Catal. 2020, 10, 114-120. (d) Zhou, T.; Jiang, M.-X.; Yang, X.; Yue, Q.; Han, Y.-Q.; Ding, Y.; Shi, B.-F. Synthesis of chiral β-lactams by Pd-catalyzed enantioselective amidation of methylene C(sp³)-H bonds. Chin. J. Chem. 2020, 38, 242-246.
(21) (a) Canty, A. J.; Jin, H.; Skelton, B. W.; White, A. H. Oxidation of complexes by (O₂CPh)₂ and (ER)₂ (E=S, Se), including structures of Pd(CH₂CH₂CH₂CH₂)(SePh)₂(bpy) (bpy=2,2′-bipyridine) and MMe₂(SePh)₂(L2) (M=Pd, Pt; L2=bpy, 1,10-phenanthroline) and C . . . O and C . . . E bond formation at palladium(IV). Inorg. Chem. 1998, 37, 3975-3981. (b) Oloo, W.; Zavalij, P. Y.; Zhang, J.; Khaskin, E.; Vedernikov, A. N. Preparation and C—X reductive elimination reactivity of monoaryl Pd^{IV}—X complexes in water (X=OH, OH₂, Cl, Br). J. Am. Chem. Soc. 2010, 132, 14400-14402. (c) Abada, E.; Zavalij, P. Y.; Vedernikov, A. N. Reductive C(sp²)-N elimination from isolated Pd(IV) amido aryl complexes prepared using H₂O₂ as oxidant. J. Am. Chem. Soc. 2017, 139, 643-646.

### EXAMPLES

Additional embodiments of the present disclosure are set forth in the following non-limiting examples.

General Information. Pd(OAc)₂, LiOAc, Ag₂CO₃, and sodium percarbonate (Na₂CO₃·1.5H₂O₂) were purchased from Sigma-Aldrich. Pd(CH₃CN)₄(BF₄)₂ was purchased from Strem. 1-Fluoro-2,4,6-trimethylpyridinium tetrafluoroborate was purchased from TCI. Hexafluoroisopropanol (HFIP) was purchased from Oakwood. Other reagents were purchased at the highest commercial quality and used without further purification, unless otherwise stated. Analytical thin layer chromatography was performed on 0.25 mm silica gel 60-F254. Visualization was carried out with short-wave UV light or KMnO₄ and heat as developing agents. ¹H NMR spectra were recorded on Bruker DRX-600 instrument. Chemical shifts were quoted in parts per million (ppm) referenced to 0.00 ppm for TMS. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 was fully decoupled by broad band proton decoupling. Chemical shifts were reported in ppm referenced to the center line of a triplet at 77.16 ppm of CDCl₃. Column chromatography was performed using E. Merck silica (60, particle size 0.043-0.063 mm), and preparative thin layer chromatography (pTLC) was performed on Merck silica plates (60E-254). High-resolution mass spectra (HRMS) were recorded on an Agilent Mass spectrometer using ESI-TOF (electrospray ionization-time of flight).

Preparation of aliphatic acids. Aliphatic carboxylic acids 1a-1w were obtained from the commercial sources or synthesized following literature procedures.¹⁻⁵

Preparation of mono-N-protected β-amino acid ligand. Ligands L5-L11 are commercially available or synthesized following literature procedures.⁶⁻⁹

### General Procedure for the Cyclative C—H/C—H Coupling Reaction

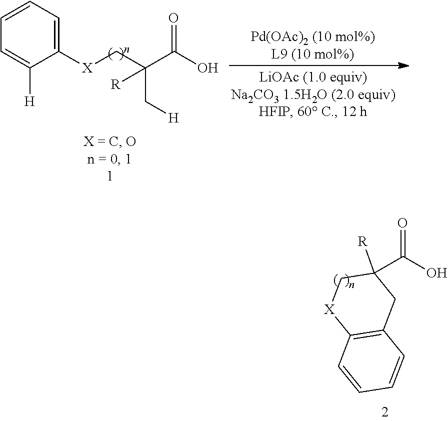

General Procedure A: In the culture tube, Pd(OAc)₂ (10 mol %, 2.2 mg), ligand L9 (10 mol %, 1.7 mg), LiOAc (1.0 equiv, 6.6 mg), Na₂CO₃·1.5H₂O₂ (2.0 equiv, 31.4 mg), and 1 (0.1 mmol) in order were weighed in air and placed with a magnetic stir bar. Then HFIP (1.0 mL) was added. The reaction mixture was stirred at rt for 3 min, and then heated to 60° C. for 12 h (600 rpm). After being allowed to cool to room temperature, the mixture was treated with HCO₂H (0.1 mL) and concentrated in vacuo. The crude mixture was purified by pTLC (hexane/EA with 1% AcOH) to afford the product 2.

General Procedure B: In the culture tube, Pd(CH₃CN)₄(BF₄)₂ (10 mol %, 4.4 mg), Ag₂CO₃ (1.0 equiv, 27.4 mg), 1-fluoro-2,4,6-trimethylpyridinium tetrafluoroborate (2.0 equiv, 45.4 mg), and 1 (0.1 mmol) in order were weighed in air and placed with a magnetic stir bar. Then HFIP (1.0 mL) was added. The reaction mixture was stirred at rt for 3 min, and then heated to 90° C. for 12 h (600 rpm). After being allowed to cool to room temperature, the mixture was treated with HCO₂H (0.1 mL), diluted with dichloromethane (DCM), filtered through a Celite plug, and concentrated in vacuo. The crude mixture was purified by pTLC (hexane/EA with 1% AcOH) to afford the product 2.

### Example 1: 2-Ethyl-1,2,3,4-tetrahydronaphthalene-2-carboxylic Acid (2a)

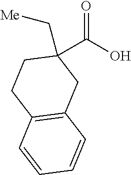 [SMILES: CCC1(C(=O)O)CCc2ccccc2C1]

Following General Procedure A on 0.1 mmol scale. Purification by pTLC afforded the title compound (colorless oil, 16.0 mg, 78% yield).

¹H NMR (600 MHz, CDCl₃) δ 7.14-7.03 (m, 4H), 3.22 (d, J=16.5 Hz, 1H), 2.92-2.83 (m, 1H), 2.83-2.75 (m, 1H), 2.67 (d, J=16.5 Hz, 1H), 2.20-2.12 (m, 1H), 1.85-1.77 (m, 1H), 1.79-1.69 (m, 1H), 1.70-1.61 (m, 1H), 0.94 (t, J=7.5 Hz, 3H).

¹³C NMR (150 MHz, CDCl₃) δ 182.5, 135.5, 134.9, 129.3, 128.8, 126.0, 125.9, 46.0, 36.6, 31.1, 30.1, 26.3, 8.9.

HRMS (ESI-TOF) Calcd for C₁₃H₁₅O₂⁻ [M−H]⁻: 203.1078; found: 203.1072.

### Example 2: 2-Ethyl-7-methyl-1,2,3,4-tetrahydronaphthalene-2-carboxylic Acid (2b)

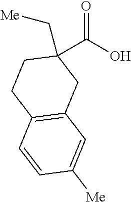 [SMILES: CCC1(C(=O)O)CCc2ccc(C)cc2C1]

Following General Procedure A on 0.1 mmol scale. Purification by pTLC afforded the title compound (colorless oil, 16.5 mg, 76% yield).

¹H NMR (600 MHz, CDCl₃) δ 7.00-6.93 (m, 1H), 6.93-6.85 (m, 2H), 3.17 (d, J=16.4 Hz, 1H), 2.87-2.78 (m, 1H), 2.78-2.70 (m, 1H), 2.63 (d, J=16.4 Hz, 1H), 2.28 (s, 3H), 2.18-2.08 (m, 1H), 1.84-1.75 (m, 1H), 1.77-1.68 (m, 1H), 1.69-1.59 (m, 1H), 0.93 (t, J=7.4 Hz, 3H).

¹³C NMR (150 MHz, CDCl₃) (major and minor rotamers) δ 182.8, 135.6, 135.6, 135.5, 134.8, 132.6, 132.0, 130.1, 129.6, 129.4, 128.9, 127.1, 127.0, 46.3, 46.2, 36.8, 36.5, 31.3, 31.3, 30.5, 30.3, 26.4, 26.1, 21.3, 9.1.

HRMS (ESI-TOF) Calcd for C₁₄H₁₇O₂⁻ [M−H]⁻: 217.1234; found: 217.1232.

### Example 3: 2-Ethyl-7-fluoro-1,2,3,4-tetrahydronaphthalene-2-carboxylic Acid (2c)

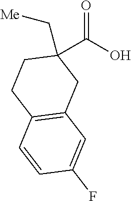 [SMILES: CCC1(C(=O)O)CCc2ccc(F)cc2C1]

Following General Procedure A on 0.1 mmol scale. Purification by pTLC afforded the title compound (colorless oil, 13.0 mg, 59% yield).

¹H NMR (600 MHz, CDCl₃) δ 7.06-6.97 (m, 1H), 6.84-6.73 (m, 2H), 3.24-3.12 (m, 1H), 2.90-2.71 (m, 2H), 2.68-2.58 (m, 1H), 2.20-2.11 (m, 1H), 1.83-1.68 (m, 2H), 1.68-1.60 (m, 1H), 0.98-0.90 (m, 3H).

¹³C NMR (150 MHz, CDCl₃) (major rotamer) δ 182.2, 161.2 (d, J=243.4 Hz), 136.9 (d, J=7.2 Hz), 130.9 (d, J=2.8 Hz), 130.1 (d, J=8.2 Hz), 115.0 (d, J=20.4 Hz), 113.1 (d, J=21.3 Hz), 45.8, 36.6, 31.3, 30.3, 25.7, 8.9.

¹³C NMR (150 MHz, CDCl₃) (minor rotamer) δ 182.3, 161.2 (d, J=243.4 Hz), 137.4 (d, J=7.2 Hz), 130.5 (d, J=7.8 Hz), 130.4 (d, J=2.9 Hz), 115.4 (d, J=20.8 Hz), 115.2 (d, J=21.0 Hz), 46.1, 36.0, 31.2, 29.8, 26.5, 8.9.

HRMS (ESI-TOF) Calcd for C₁₃H₁₄FO₂⁻ [M−H]⁻: 221.0983; found: 221.0990.

### Example 4: 7-Chloro-2-ethyl-1,2,3,4-tetrahydronaphthalene-2-carboxylic Acid (2d)

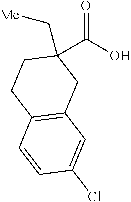 [SMILES: CCC1(C(=O)O)CCc2ccc(Cl)cc2C1]

Following General Procedure A on 0.1 mmol scale. Purification by pTLC afforded the title compound (colorless oil, 14.5 mg, 61% yield).

¹H NMR (600 MHz, CDCl₃) δ 7.15-7.06 (m, 2H), 7.06-6.98 (m, 1H), 3.25-3.15 (m, 1H), 2.90-2.73 (m, 2H), 2.69-2.59 (m, 1H), 2.22-2.13 (m, 1H), 1.85-1.71 (m, 2H), 1.71-1.61 (m, 1H), 0.93 (t, J=7.5 Hz, 3H).

¹³C NMR (150 MHz, CDCl₃) (major and minor rotamers) δ 182.0, 182.0, 137.3, 136.8, 133.9, 133.4, 131.4, 131.4, 130.6, 130.1, 129.0, 128.6, 126.1, 126.1, 46.0, 45.8, 36.4, 36.1, 31.3, 31.3, 30.1, 29.9, 26.3, 25.8, 8.9.

HRMS (ESI-TOF) Calcd for C₁₃H₁₄ClO₂⁻ [M−H]⁻: 237.0688; found: 237.0684.

### Example 5: 2-Ethyl-1,2,3,4-tetrahydrophenanthrene-2-carboxylic Acid (2e)

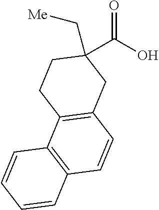 [SMILES: CCC1(C(=O)O)CCc2c(ccc3ccccc23)C1]

Following General Procedure A on 0.1 mmol scale. Purification by pTLC afforded the title compound (colorless oil, 13.3 mg, 52% yield).

¹H NMR (600 MHz, CDCl₃) δ 7.92 (d, J=8.4 Hz, 1H), 7.78 (d, J=8.1 Hz, 1H), 7.62 (d, J=8.4 Hz, 1H), 7.51-7.45 (m, 1H), 7.45-7.40 (m, 1H), 7.20 (d, J=8.4 Hz, 1H), 3.35 (d, J=16.7 Hz, 1H), 3.23-3.12 (m, 2H), 2.82 (d, J=16.7 Hz, 1H), 2.36-2.29 (m, 1H), 1.99-1.91 (m, 1H), 1.83-1.74 (m, 1H), 1.74-1.66 (m, 1H), 0.97 (t, J=7.5 Hz, 3H).

¹³C NMR (151 MHz, CDCl₃) δ 182.5, 132.3, 132.2, 132.1, 130.1, 128.6, 128.2, 126.3, 126.1, 125.0, 123.0, 45.7, 37.5, 30.9, 29.8, 23.2, 9.0.

HRMS (ESI-TOF) Calcd for C₁₇H₁₇O₂⁻ [M−H]⁻: 253.1234; found: 253.1230.

### Example 6: 2-Methyl-1,2,3,4-tetrahydronaphthalene-2-carboxylic Acid (2f)

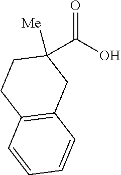 [SMILES: CC1(C(=O)O)CCc2ccccc2C1]

Following General Procedure A on 0.1 mmol scale. Purification by pTLC afforded the title compound (colorless oil, 12.5 mg, 66% yield).

¹H NMR (600 MHz, CDCl₃) δ 7.17-7.02 (m, 4H), 3.24 (d, J=16.4 Hz, 1H), 2.95-2.86 (m, 1H), 2.87-2.78 (m, 1H), 2.67 (d, J=16.4 Hz, 1H), 2.21-2.13 (m, 1H), 1.85-1.75 (m, 1H), 1.32 (s, 3H).

¹³C NMR (150 MHz, CDCl₃) δ 182.7, 135.1, 134.7, 129.4, 128.9, 126.0, 126.0, 41.6, 38.5, 31.8, 26.2, 24.4.

HRMS (ESI-TOF) Calcd for C₁₂H₁₃O₂⁻ [M−H]⁻: 189.0921; found: 189.0919.

### Example 7: 7-Fluoro-2-methyl-1,2,3,4-tetrahydronaphthalene-2-carboxylic Acid (2g)

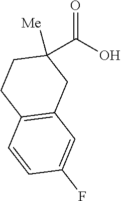 [SMILES: CC1(C(=O)O)CCc2ccc(F)cc2C1]

Following General Procedure A on 0.1 mmol scale. Purification by pTLC afforded the title compound (colorless oil, 11.0 mg, 53% yield).

¹H NMR (600 MHz, CDCl₃) δ 7.06-6.99 (m, 1H), 6.84-6.74 (m, 2H), 3.26-3.14 (m, 1H), 2.93-2.74 (m, 2H), 2.67-2.57 (m, 1H), 2.22-2.12 (m, 1H), 1.81-1.72 (m, 1H), 1.31 (s, 3H).

¹³C NMR (150 MHz, CDCl₃) (major rotamer) δ 183.1, 161.2 (d, J=243.6 Hz), 136.7 (d, J=7.3 Hz), 130.5 (d, J=1.8 Hz), 130.2 (d, J=7.8 Hz), 115.4 (d, J=20.8 Hz), 113.2 (d, J=21.1 Hz), 41.5, 38.5, 31.9, 25.6, 24.5.

¹³C NMR (150 MHz, CDCl₃) (minor rotamer) δ 183.2, 161.2 (d, J=243.6 Hz), 137.0 (d, J=7.2 Hz), 130.6 (d, J=6.2 Hz), 130.2 (d, J=3.1 Hz), 115.0 (d, J=20.5 Hz), 113.1 (d, J=21.3 Hz), 41.7, 37.8, 31.5, 26.5, 24.5.

HRMS (ESI-TOF) Calcd for C₁₂H₁₂FO₂⁻ [M−H]⁻: 207.0827; found: 207.0825.

### Example 8: 2-Butyl-1,2,3,4-tetrahydronaphthalene-2-carboxylic Acid (2h)

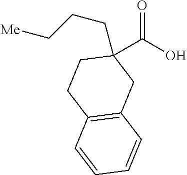 [SMILES: CCCCC1(C(=O)O)CCc2ccccc2C1]

Following General Procedure A on 0.1 mmol scale. Purification by pTLC afforded the title compound (colorless oil, 16.5 mg, 71% yield).

¹H NMR (600 MHz, CDCl₃) δ 7.13-7.03 (m, 4H), 3.22 (d, J=16.4 Hz, 1H), 2.91-2.82 (m, 1H), 2.82-2.74 (m, 1H), 2.69 (d, J=16.4 Hz, 1H), 2.20-2.10 (m, 1H), 1.87-1.77 (m, 1H), 1.73-1.63 (m, 1H), 1.63-1.55 (m, 1H), 1.35-1.23 (m, 4H), 0.89 (t, J=6.8 Hz, 3H).

¹³C NMR (150 MHz, CDCl₃) δ 181.4, 135.3, 134.7, 129.1, 128.6, 125.7, 125.7, 45.3, 37.9, 37.0, 30.2, 26.5, 26.1, 23.0, 13.9.

HRMS (ESI-TOF) Calcd for C₁₅H₁₉O₂⁻ [M−H]⁻: 231.1391; found: 231.1390.

### Example 9: 1,2,3,4-Tetrahydronaphthalene-2-carboxylic Acid (2i)

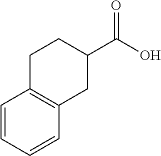 [SMILES: O=C(O)C1CCc2ccccc2C1]

Following General Procedure A on 0.1 mmol scale. Purification by pTLC afforded the title compound (colorless oil, 11.5 mg, 65% yield).

¹H NMR (600 MHz, CDCl₃) δ 7.17-7.03 (m, 4H), 3.11-2.97 (m, 2H), 2.95-2.84 (m, 2H), 2.84-2.75 (m, 1H), 2.29-2.20 (m, 1H), 1.96-1.83 (m, 1H).

¹³C NMR (150 MHz, CDCl₃) δ 181.7, 135.7, 134.7, 129.2, 129.0, 126.2, 126.0, 39.9, 31.5, 28.5, 25.8.

HRMS (ESI-TOF) Calcd for C₁₁H₁₁O₂⁻ [M−H]⁻: 175.0765; found: 175.0757.

The NMR data matches the reported data¹¹.

### Example 10: 6-Methoxy-1,2,3,4-tetrahydronaphthalene-2-carboxylic Acid (2j)

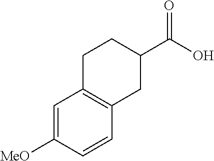 [SMILES: COc1ccc2c(c1)CCC(C(=O)O)C2]

Following General Procedure A on 0.1 mmol scale. Purification by pTLC afforded the title compound (colorless oil, 12.0 mg, 58% yield).

¹H NMR (600 MHz, CDCl₃) δ 7.02 (d, J=8.4 Hz, 1H), 6.71 (d, J=8.4 Hz, 1H), 6.63 (s, 1H), 3.77 (s, 3H), 3.05-2.93 (m, 2H), 2.91-2.83 (m, 2H), 2.82-2.73 (m, 1H), 2.29-2.19 (m, 1H), 1.94-1.82 (m, 1H).

¹³C NMR (150 MHz, CDCl₃) δ 181.2, 157.9, 136.8, 130.1, 126.8, 113.6, 112.4, 55.4, 40.1, 30.7, 28.8, 25.7.

HRMS (ESI-TOF) Calcd for C₁₂H₁₃O₃⁻ [M−H]⁻: 205.0870; found: 205.0869.

### Example 11: 5-Methoxy-1,2,3,4-tetrahydronaphthalene-2-carboxylic Acid (2k)

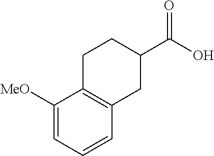 [SMILES: COc1cccc2c1CCC(C(=O)O)C2]

Following General Procedure A on 0.1 mmol scale. Purification by pTLC afforded the title compound (colorless oil, 7.3 mg, 35% yield).

¹H NMR (600 MHz, CDCl₃) δ 7.10 (t, J=7.9 Hz, 1H), 6.72 (d, J=7.7 Hz, 1H), 6.67 (d, J=8.1 Hz, 1H), 3.82 (s, 3H), 3.18-3.08 (m, 1H), 2.93-2.80 (m, 2H), 2.79-2.70 (m, 2H), 2.25-2.18 (m, 1H), 1.92-1.78 (m, 1H).

¹³C NMR (150 MHz, CDCl₃) δ 179.1, 157.5, 137.1, 126.4, 123.7, 121.1, 107.2, 55.4, 39.4, 28.7, 25.6, 25.4.

HRMS (ESI-TOF) Calcd for C₁₂H₁₃O₃⁻ [M−H]⁻: 205.0870; found: 205.0869.

### Example 12: 7-Fluoro-1,2,3,4-tetrahydronaphthalene-2-carboxylic Acid (2l)

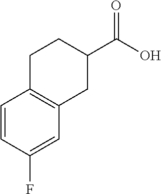 [SMILES: O=C(O)C1CCc2ccc(F)cc2C1]

Following General Procedure A on 0.1 mmol scale. Purification by pTLC afforded the title compound (colorless oil, 10.9 mg, 56% yield).

¹H NMR (600 MHz, CDCl₃) δ 7.11-7.03 (m, 1H), 6.91-6.78 (m, 2H), 3.12-2.96 (m, 2H), 2.95-2.88 (m, 1H), 2.87-2.76 (m, 2H), 2.33-2.21 (m, 1H), 1.97-1.87 (m, 1H).

¹³C NMR (150 MHz, CDCl₃) (major rotamer) δ 180.7, 161.2 (d, J=243.7 Hz), 136.6 (d, J=7.4 Hz), 131.2 (d, J=2.7 Hz), 130.3 (d, J=8.2 Hz), 115.3 (d, J=20.6 Hz), 113.3 (d, J=21.4 Hz), 39.5, 31.4, 27.8, 25.8.

¹³C NMR (150 MHz, CDCl₃) (minor rotamer) δ 180.8, 161.3 (d, J=244.2 Hz), 137.6 (d, J=7.3 Hz), 130.5 (d, J=7.8 Hz), 130.2 (d, J=2.8 Hz), 115.1 (d, J=20.7 Hz), 113.2 (d, J=21.1 Hz), 39.7, 30.8, 28.6, 25.4.

HRMS (ESI-TOF) Calcd for C₁₁H₁₀FO₂⁻ [M−H]⁻: 193.0670; found: 193.0666.

### Example 13: 3-Methylchromane-3-carboxylic Acid (2m)

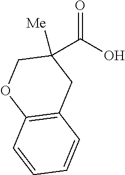 [SMILES: CC1(C(=O)O)COc2ccccc2C1]

Following General Procedure A on 0.1 mmol scale. Purification by pTLC afforded the title compound (colorless oil, 13.0 mg, 68% yield).

¹H NMR (600 MHz, CDCl₃) δ 7.15-7.08 (m, 1H), 7.06 (d, J=7.4 Hz, 1H), 6.91-6.85 (m, 1H), 6.83 (d, J=8.2 Hz, 1H), 4.31 (dd, J=10.8, 1.4 Hz, 1H), 3.95 (d, J=10.8 Hz, 1H), 3.27 (d, J=16.4 Hz, 1H), 2.70 (d, J=16.4 Hz, 1H), 1.34 (s, 3H).

¹³C NMR (150 MHz, CDCl₃) δ 180.7, 153.5, 130.0, 127.7, 121.1, 120.1, 116.8, 71.0, 40.8, 34.5, 21.1.

HRMS (ESI-TOF) Calcd for C₁₁H₁₁O₃⁻ [M−H]⁻: 191.0714; found: 191.0713.

The NMR data matches the reported data¹².

### Example 14: 7-(tert-Butyl)-3-methylchromane-3-carboxylic Acid (2n)

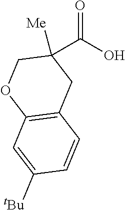 [SMILES: CC1(C(=O)O)COc2cc(C(C)(C)C)ccc2C1]

Following General Procedure A on 0.1 mmol scale. Purification by pTLC afforded the title compound (colorless oil, 20.0 mg, 80% yield, 2n/2n′=3/1).

¹H NMR (600 MHz, CDCl₃) δ 6.99 (d, J=8.0 Hz, 1H), 6.92 (dd, J=8.0, 2.0 Hz, 1H), 6.86 (d, J=2.0 Hz, 1H), 4.29 (dd, J=10.8, 1.4 Hz, 1H), 3.93 (dd, J=10.8, 1.4 Hz, 1H), 3.24 (d, J=16.3 Hz, 1H), 2.66 (d, J=16.3 Hz, 1H), 1.34 (s, 3H), 1.28 (s, 9H).

¹³C NMR (150 MHz, CDCl₃) δ 180.8, 153.0, 151.2, 129.4, 118.4, 117.0, 113.7, 71.0, 40.9, 34.6, 34.1, 31.4, 21.2.

HRMS (ESI-TOF) Calcd for C₁₅H₁₉O₃⁻ [M−H]⁻: 247.1340; found: 247.1339.

### Example 15: 5-(tert-Butyl)-3-methylchromane-3-carboxylic Acid (2n′)

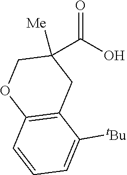 [SMILES: CC1(C(=O)O)COc2cccc(C(C)(C)C)c2C1]

¹H NMR (600 MHz, CDCl₃) δ 7.05 (t, J=7.8 Hz, 1H), 6.99 (d, J=7.8 Hz, 1H), 6.73 (d, J=7.8 Hz, 1H), 4.37 (d, J=10.5 Hz, 1H), 3.91 (d, J=10.5 Hz, 1H), 3.51 (d, J=16.0 Hz, 1H), 2.90 (d, J=16.0 Hz, 1H), 1.42 (s, 9H), 1.35 (s, 3H).

¹³C NMR (150 MHz, CDCl₃) δ 180.9, 154.0, 149.4, 127.1, 119.0, 118.9, 115.6, 70.4, 40.8, 36.2, 34.9, 31.2, 21.5.

HRMS (ESI-TOF) Calcd for C₁₅H₁₉O₃⁻ [M−H]⁻: 247.1340; found: 247.1337.

### Example 16: 3-Methyl-3,4,7,8,9,10-hexahydro-2H-benzo[h]chromene-3-carboxylic Acid (2o)

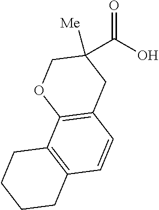 [SMILES: CC1(C(=O)O)COc2c(ccc3c2CCCC3)C1]

Following General Procedure A on 0.1 mmol scale. Purification by pTLC afforded the title compound (colorless oil, 21.0 mg, 85% yield).

¹H NMR (600 MHz, CDCl₃) δ 6.81 (d, J=7.8 Hz, 1H), 6.63 (d, J=7.8 Hz, 1H), 4.29 (d, J=10.8 Hz, 1H), 3.96 (d, J=10.8 Hz, 1H), 3.23 (d, J=16.3 Hz, 1H), 2.70 (t, J=5.8 Hz, 2H), 2.65 (d, J=16.3 Hz, 1H), 2.64-2.58 (m, 2H), 1.80-1.69 (m, 4H), 1.33 (s, 3H).

¹³C NMR (150 MHz, CDCl₃) δ 181.2, 151.1, 136.7, 126.5, 125.4, 121.6, 116.2, 70.9, 40.7, 34.5, 29.6, 23.1, 23.0, 22.9, 21.1.

HRMS (ESI-TOF) Calcd for C₁₅H₁₇O₃⁻ [M−H]⁻: 245.1183; found: 245.1183.

### Example 17: 8-Benzyl-3-methylchromane-3-carboxylic Acid (2p)

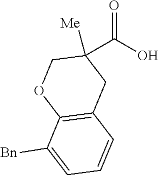 [SMILES: CC1(C(=O)O)COc2c(Cc3ccccc3)cccc2C1]

Following General Procedure A on 0.1 mmol scale. Purification by pTLC afforded the title compound (colorless oil, 20.0 mg, 70% yield).

¹H NMR (600 MHz, CDCl₃) δ 7.27-7.20 (m, 2H), 7.19 (d, J=7.5 Hz, 2H), 7.15 (t, J=7.3 Hz, 1H), 6.94 (d, J=7.5 Hz, 1H), 6.89 (d, J=7.4 Hz, 1H), 6.80 (t, J=7.5 Hz, 1H), 4.31 (d, J=10.7 Hz, 1H), 4.03-3.88 (m, 3H), 3.28 (d, J=16.4 Hz, 1H), 2.71 (d, J=16.4 Hz, 1H), 1.34 (s, 3H).

¹³C NMR (150 MHz, CDCl₃) δ 180.5, 151.2, 141.1, 129.1, 129.0, 128.5, 128.4, 128.1, 125.9, 120.7, 119.9, 71.0, 40.7, 35.7, 34.7, 21.0.

HRMS (ESI-TOF) Calcd for C₁₈H₁₇O₃⁻ [M−H]⁻: 281.1183; found: 281.1184.

### Example 18: 8-Bromo-3-methylchromane-3-carboxylic Acid (2q)

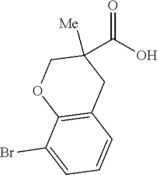 [SMILES: CC1(C(=O)O)COc2c(Br)cccc2C1]

Following General Procedure A on 0.1 mmol scale. Purification by pTLC afforded the title compound (colorless oil, 8.5 mg, 31% yield).

¹H NMR (600 MHz, CDCl₃) δ 7.36 (d, J=7.8 Hz, 1H), 7.02 (d, J=7.8 Hz, 1H), 6.76 (t, J=7.8 Hz, 1H), 4.41 (d, J=10.8 Hz, 1H), 4.07 (d, J=10.8 Hz, 1H), 3.29 (d, J=16.4 Hz, 1H), 2.72 (d, J=16.4 Hz, 1H), 1.36 (s, 3H).

¹³C NMR (151 MHz, CDCl₃) δ 179.8, 150.1, 131.5, 129.2, 121.9, 110.9, 71.7, 40.7, 34.6, 21.0 (1 carbon signal was not assigned due to overlaps).

HRMS (ESI-TOF) Calcd for C₁₁H₁₀BrO₃⁻ [M−H]⁻: 268.9819; found: 268.9820.

### Example 19: 3-Methyl-8-(trifluoromethyl)chromane-3-carboxylic Acid (2r)

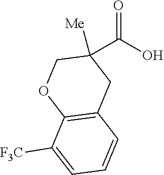 [SMILES: CC1(C(=O)O)COc2c(cccc2C(F)(F)F)C1]

Following General Procedure A on 0.1 mmol scale. Purification by pTLC afforded the title compound (colorless oil, 6.0 mg, 23% yield).

¹H NMR (600 MHz, CDCl₃) δ 7.40 (d, J=7.7 Hz, 1H), 7.23 (d, J=7.7 Hz, 1H), 6.93 (t, J=7.7 Hz, 1H), 4.38 (d, J=11.3 Hz, 1H), 4.06 (d, J=11.3 Hz, 1H), 3.29 (d, J=16.4 Hz, 1H), 2.73 (d, J=16.4 Hz, 1H), 1.35 (s, 3H).

¹³C NMR (150 MHz, CDCl₃) δ 179.5, 151.6, 133.8, 125.4 (q, J=5.4 Hz), 123.7 (q, J=272.3 Hz), 121.6, 120.2, 118.2 (q, J=30.9 Hz), 71.2, 40.3, 34.3, 21.0.

HRMS (ESI-TOF) Calcd for C₁₂H₁₀F₃O₃⁻ [M−H]⁻: 259.0588; found: 259.0587.

### Example 20: (R)-7-Methoxychromane-3-carboxylic Acid (2s)

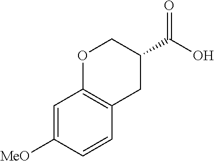 [SMILES: COc1ccc2c(c1)OC[C@H](C(=O)O)C2]

Following General Procedure A on 0.1 mmol scale. Purification by pTLC afforded the title compound (colorless oil, 15.0 mg, 72% yield).

¹H NMR (600 MHz, CDCl₃) δ 6.98 (d, J=8.4 Hz, 1H), 6.49 (dd, J=8.4, 2.6 Hz, 1H), 6.39 (d, J=2.6 Hz, 1H), 4.47-4.40 (m, 1H), 4.21-4.14 (m, 1H), 3.75 (s, 3H), 3.10-3.04 (m, 1H), 3.03-2.96 (m, 2H).

¹³C NMR (150 MHz, CDCl₃) δ 176.8, 159.4, 154.8, 130.3, 112.1, 108.1, 101.7, 66.3, 55.5, 38.4, 26.8.

HRMS (ESI-TOF) Calcd for C₁₁H₁₁O₄⁻ [M−H]⁻: 207.0663; found: 207.0660.

### Example 21: 2-Ethyl-2,3-dihydro-1H-indene-2-carboxylic Acid (2u)

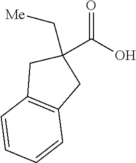 [SMILES: CCC1(C(=O)O)Cc2ccccc2C1]

Following General Procedure A on 0.1 mmol scale. Purification by pTLC afforded the title compound (colorless oil, 10.0 mg, 53% yield).

¹H NMR (600 MHz, CDCl₃) δ 7.21-7.16 (m, 2H), 7.16-7.11 (m, 2H), 3.48 (d, J=16.2 Hz, 2H), 2.92 (d, J=16.2 Hz, 2H), 1.83 (q, J=7.2 Hz, 2H), 0.94 (t, J=7.2 Hz, 3H).

¹³C NMR (150 MHz, CDCl₃) δ 182.3, 141.4, 126.7, 124.6, 54.7, 41.8, 31.5, 10.0.

HRMS (ESI-TOF) Calcd for C₁₂H₁₃O₂⁻ [M−H]⁻: 189.0921; found: 189.0918.

### Example 22: 2,4-Dimethyl-2,3-dihydro-1H-indene-2-carboxylic Acid (2v)

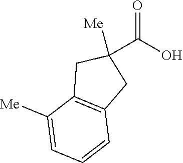 [SMILES: Cc1cccc2c1CC(C)(C(=O)O)C2]

Following General Procedure B on 0.1 mmol scale. Purification by pTLC afforded the title compound (colorless oil, 11.5 mg, 61% yield).

¹H NMR (600 MHz, CDCl₃) δ 7.08 (t, J=7.4 Hz, 1H), 7.03 (d, J=7.4 Hz, 1H), 6.98 (d, J=7.4 Hz, 1H), 3.53 (d, J=15.9 Hz, 1H), 3.43 (d, J=16.0 Hz, 1H), 2.86 (d, J=15.9 Hz, 1H), 2.80 (d, J=16.0 Hz, 1H), 2.24 (s, 3H), 1.41 (s, 3H).

¹³C NMR (150 MHz, CDCl₃) δ 184.2, 141.0, 140.0, 134.2, 127.6, 127.0, 122.1, 49.0, 44.2, 42.8, 25.4, 19.2.

HRMS (ESI-TOF) Calcd for C₁₂H₁₃O₂⁻ [M−H]⁻: 189.0921; found: 189.0915.

### Example 23: 2-Methyl-2,3-dihydro-1H-indene-2-carboxylic Acid (2w)

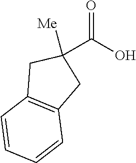 [SMILES: CC1(C(=O)O)Cc2ccccc2C1]

Following General Procedure B on 0.1 mmol scale. Purification by pTLC afforded the title compound (colorless oil, 8.0 mg, 48% yield).

¹H NMR (600 MHz, CDCl₃) δ 7.23 — 7.18 (m, 2H), 7.18 — 7.14 (m, 2H), 3.52 (d, J=15.8 Hz, 2H), 2.85 (d, J=15.8 Hz, 2H), 1.41 (s, 3H).

¹³C NMR (150 MHz, CDCl₃) δ 182.5, 141.2, 126.8, 124.8, 49.5, 44.0, 25.0.

HRMS (ESI-TOF) Calcd for C₁₁H₁₁O₂⁻ [M−H]⁻: 175.0765; found: 175.0762.

The NMR data matches the reported data¹³.

### Example 24: Total Synthesis of (±)-russujaponol F

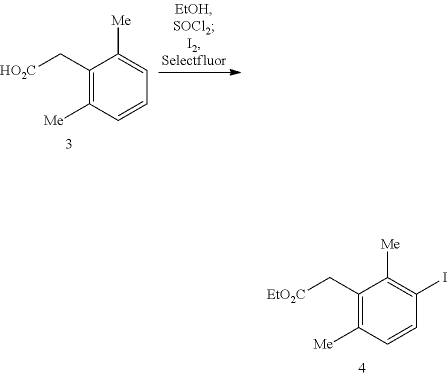 [SMILES: Cc1cccc(C)c1CC(=O)O ; CCOC(=O)Cc1c(C)ccc(I)c1C]

To an EtOH (5.0 mL) solution of 3 (1.0 mmol, 164 mg) was added SOCl₂ (2.0 equiv, 0.15 mL) at 0° C. and then the mixture was stirred under reflux overnight. After being allowed to cool to room temperature, the mixture was concentrated in vacuo to afford the corresponding ethyl ester. Following literature procedure with slight modification, to the CH₃CN solution (10.0 mL) of the ethyl ester was added I₂ (0.5 equiv, 127 mg) and Selectfluor (0.5 equiv, 177 mg) and the mixture was stirred at 60° C. for 3 h. After being allowed to cool to room temperature, the mixture was diluted with EA, washed with saturated Na₂S₂O₃, and concentrated in vacuo. The crude mixture was purified by column chromatography to afford the iodination product 4 (250 mg, 79% yield).

### Ethyl 2-(3-iodo-2,6-dimethylphenyl)acetate (4)

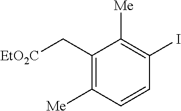 [SMILES: CCOC(=O)Cc1c(C)ccc(I)c1C]

¹H NMR (600 MHz, CDCl₃) δ 7.65 (d, J=8.1 Hz, 1H), 6.74 (d, J=8.1 Hz, 1H), 4.15 (q, J=7.1 Hz, 2H), 3.75 (s, 2H), 2.48 (s, 3H), 2.29 (s, 3H), 1.25 (t, J=7.1 Hz, 3H).

¹³C NMR (150 MHz, CDCl₃) δ 171.0, 139.9, 138.1, 137.8, 133.0, 129.8, 99.7, 61.1, 37.1, 26.0, 20.5, 14.3.

HRMS (ESI-TOF) Calcd for C₁₂H₁₆IO₂⁺ [M+H]⁺: 319.0189; found: 319.0196.

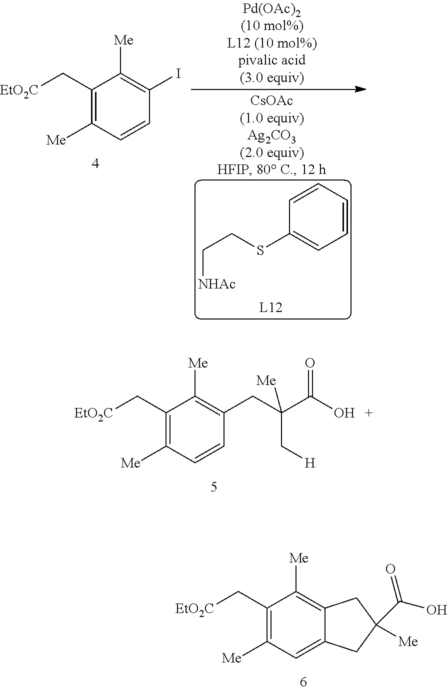 [SMILES: CCOC(=O)Cc1c(C)ccc(I)c1C ; CC(=O)NCCSc1ccccc1 ; CCOC(=O)Cc1c(C)ccc(CC(C)(C)C(=O)O)c1C ; CCOC(=O)Cc1c(C)cc2c(c1C)CC(C)(C(=O)O)C2]

In a culture tube, Pd(OAc)₂ (10 mol %, 2.2 mg), ligand L12 (10 mol %, 2.0 mg), CsOAc (1.0 equiv, 19.2 mg), Ag₂CO₃ (2.0 equiv, 55.1 mg), pivalic acid (3.0 equiv, 30.6 mg) and 4 (0.1 mmol, 31.8 mg) in order were weighed in air and placed with a magnetic stir bar. Then HFIP (1.0 mL) was added. The reaction mixture was stirred at rt for 3 min, and then heated to 80° C. for 12 h (600 rpm). After being allowed to cool to room temperature, the mixture was treated with HCO₂H (0.1 mL), diluted with DCM, filtered through a Celite plug, and concentrated in vacuo. The crude mixture was purified by pTLC (hexane/EA) to afford the arylation product 5 (18.0 mg, 62% yield) and the product 6 (3.5 mg, 12% yield).

### 3-(3-(2-Ethoxy-2-oxoethyl)-2,4-dimethylphenyl)-2,2-dimethylpropanoic Acid (5)

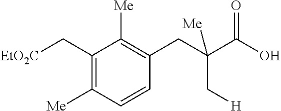 [SMILES: CCOC(=O)Cc1c(C)ccc(CC(C)(C)C(=O)O)c1C]

¹H NMR (600 MHz, CDCl₃) δ 6.99 (d, J=7.9 Hz, 1H), 6.96 (d, J=7.9 Hz, 1H), 4.14 (q, J=7.1 Hz, 2H), 3.70 (s, 2H), 2.99 (s, 2H), 2.30 (s, 3H), 2.26 (s, 3H), 1.23 (t, J=7.1 Hz, 3H), 1.19 (s, 6H).

¹³C NMR (150 MHz, CDCl₃) δ 183.1, 171.6, 136.5, 135.7, 134.0, 132.5, 130.1, 127.5, 60.9, 44.1, 42.3, 36.2, 27.3, 24.7, 20.7, 17.0, 14.4.

HRMS (ESI-TOF) Calcd for C₁₇H₂₃O₄⁻ [M−H]⁻: 291.1602; found: 291.1605.

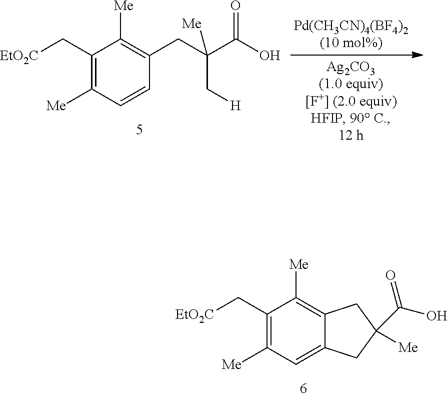 [SMILES: CCOC(=O)Cc1c(C)ccc(CC(C)(C)C(=O)O)c1C ; CCOC(=O)Cc1c(C)cc2c(c1C)CC(C)(C(=O)O)C2]

In a culture tube, Pd(CH₃CN)₄(BF₄)₂ (10 mol %, 2.2 mg), Ag₂CO₃ (1.0 equiv, 13.8 mg), 1-fluoro-2,4,6-trimethylpyridinium tetrafluoroborate (2.0 equiv, 22.7 mg), and 5 (0.05 mmol, 14.6 mg) in order were weighed in air and placed with a magnetic stir bar. Then HFIP (0.5 mL) was added. The reaction mixture was stirred at rt for 3 min, and then heated to 90° C. for 12 h (600 rpm). After being allowed to cool to room temperature, the mixture was treated with HCO₂H (0.05 mL), diluted with DCM, filtered through a Celite plug, and concentrated in vacuo. The crude mixture was purified by pTLC (hexane/EA) to afford the product 6 (6.0 mg, 41% yield).

### 5-(2-Ethoxy-2-oxoethyl)-2,4,6-trimethyl-2,3-dihydro-1H-indene-2-carboxylic Acid (6)

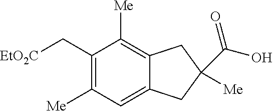 [SMILES: CCOC(=O)Cc1c(C)cc2c(c1C)CC(C)(C(=O)O)C2]

¹H NMR (600 MHz, CDCl₃) δ 6.90 (s, 1H), 4.14 (q, J=7.0 Hz, 2H), 3.66 (s, 2H), 3.49 (d, J=16.0 Hz, 1H), 3.44 (d, J=16.0 Hz, 1H), 2.81 (d, J=16.0 Hz, 1H), 2.80 (d, J=16.0 Hz, 1H), 2.30 (s, 3H), 2.21 (s, 3H), 1.41 (s, 3H), 1.25 (t, J=7.1 Hz, 3H).

¹³C NMR (150 MHz, CDCl₃) δ 181.9, 171.7, 139.7, 138.3, 136.0, 133.3, 130.0, 124.1, 60.9, 48.8, 44.2, 43.5, 35.4, 25.5, 20.8, 16.5, 14.4.

HRMS (ESI-TOF) Calcd for C₁₇H₂₁O₄⁻ [M−H]⁻: 289.1445; found: 289.1447.

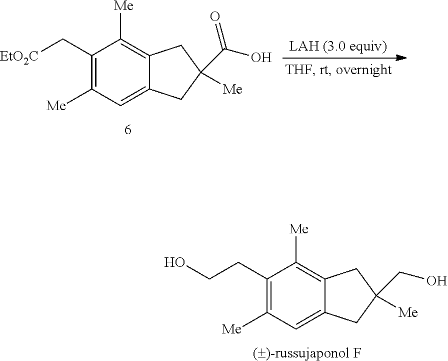 [SMILES: CCOC(=O)Cc1c(C)cc2c(c1C)CC(C)(C(=O)O)C2 ; Cc1cc2c(c(C)c1CCO)CC(C)(CO)C2]

In the culture tube, to the THF (1.0 mL) solution of 6 (0.02 mmol, 6.0 mg) was added LAH (3.0 equiv, 1.0 M in THF, 0.06 mL) at 0° C. The reaction mixture was warmed to rt and stirred at rt overnight. The mixture was diluted with ether, washed with saturated NH₄Cl, and concentrated in vacuo. The crude mixture was purified by pTLC (hexane/EA) to afford the (±)-russujaponol F (4.5 mg, 96% yield). The NMR data matches the reported data^{14,15}.

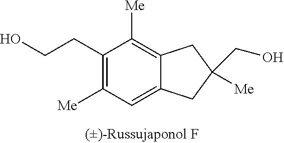 [SMILES: Cc1cc2c(c(C)c1CCO)CC(C)(CO)C2]

¹H NMR (600 MHz, CDCl₃) δ 6.87 (s, 1H), 3.74 (t, J=7.4 Hz, 2H), 3.52 (s, 2H), 2.95 (t, J=7.5 Hz, 2H), δ 2.88 (d, J=15.9 Hz, 1H), 2.84 (d, J=15.9 Hz, 1H), 2.63 (d, J=15.9 Hz, 1H), 2.59 (d, J=15.9 Hz, 1H), 2.32 (s, 3H), 2.22 (s, 3H), 1.18 (s, 3H).

¹³C NMR (150 MHz, CDCl₃) δ 140.3, 139.8, 135.4, 133.2, 132.3, 124.4, 71.1, 62.1, 44.3, 43.1, 42.4, 32.9, 24.6, 20.6, 16.3.

HRMS (ESI-TOF) Calcd for C₁₅H₂₁O₂⁻ [M−H]⁻: 233.1547; found: 233.1544.

Numbered references throughout the examples above are as follows:
1. Park, H.; Chekshin, N.; Shen, P.-X.; Yu, J.-Q. Ligand-enabled, palladium-catalyzed β-C(sp3)-H arylation of weinreb amides. ACS Catal. 2018, 8, 9292-9297.
2. Shen, P.-X.; Hu, L.; Shao, Q.; Hong, K.; Yu, J.-Q. Pd(II)-catalyzed enantioselective C(sp3)-H arylation of free carboxylic acids. J. Am. Chem. Soc. 2018, 140, 6545-6549.
3. Fillion, E.; Dumas, A. M. Synthesis of fused 4,5-disubstituted indole ring systems by intramolecular Friedel-Crafts acylation of 4-substituted indoles. J. Org. Chem. 2008, 73, 2920-2923.
4. Quach, T. D.; Batey, R. A. Copper(II)-catalyzed ether synthesis from aliphatic alcohols and potassium organotrifluoroborate salts. Org. Lett. 2003, 5, 1381-1384.
5. Ikeda, K.; Achiwa, K.; Sekiya, M. Trifluoromethanesulfonic acid-promoted reaction of hexahydro-1,3,5-triazines. Introduction of a secondary aminomethyl grouping into carboxylates at the α-position through ketene silyl acetals. Chem. Pharm. Bull. 1986, 34, 1579-1583.
6. Hong, K.; Park, H.; Yu, J.-Q. Methylene C(sp3)-H arylation of aliphatic ketones using a transient directing group. ACS Catal. 2017, 7, 6938-6941.
7. Naturale, G.; Lamblin, M.; Commandeur, C.; Felpin, F.-X.; Dessolin, J. Direct C—H alkylation of naphthoquinones with amino acids through a revisited Kochi-Anderson radical decarboxylation: trends in reactivity and applications. Eur. J. Org. Chem. 2012, 5774-5788.
8. Dener, J. M.; Fantauzzi, P. P.; Kshirsagar, T. A.; Kelly, D. E.; Wolfe, A. B. Large-scale syntheses of FMOC-protected non-proteogenic amino acids: useful building blocks for combinatorial libraries. Org. Process Res. Dev. 2001, 54, 445-449.
9. F. Fülöp, M. Palkó, J. Kámán, L. Lázár, R. Sillanpää, Synthesis of all four enantiomers of 1-aminoindane-2-carboxylic acid, a new cispentacin benzologue. Tetrahedron: Asymmetry 2000, 11, 4179-4187.
10. Stavber, S.; Kralj, P.; Zupan, M. Selective and effective iodination of alkyl-substituted benzenes with elemental iodine activated by Selectfluor™ F-TEDA-BF4. Synlett 2002, 598-600.
11. Seo, H.; Liu, A.; Jamison, T. F. Direct β-selective hydrocarboxylation of styrenes with CO2 enabled by continuous flow photoredox catalysis. J. Am. Chem. Soc. 2017, 139, 13969-13972.
12. Feng, Y. et al. Benzopyrans and analogs as Rho kinase inhibitors and their preparation and use in the treatment of Rho kinase-mediated diseases. PCT Int. Appl., 2009079008, 25 Jun. 2009.
13. Alkayal, A.; Tabas, V.; Montanaro, S.; Wright, I. A.; Malkov, A. V.; Buckley, B. R. Harnessing applied potential: selective β-hydrocarboxylation of substituted olefins. J. Am. Chem. Soc. 2020, 142, 1780-1785.
14. Melot, R.; Craveiro, M.; Btirgi, T.; Baudoin, O. Divergent enantioselective synthesis of (nor)illudalane sesquiterpenes via Pd0-catalyzed asymmetric C(sp3)-H activation. Org. Lett. 2019, 21, 812-815.
15. Melot, R.; Craveiro, M. V.; Baudoin, O. Total synthesis of (nor)illudalane sesquiterpenes based on a C(sp3)-H activation strategy. J. Org. Chem. 2019, 84, 12933-12945.

We claim:

1. A process for making a compound of formula (2): 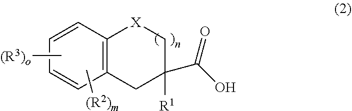
comprising contacting a compound of formula (1): 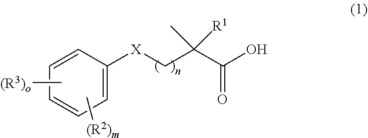
with a ligand of formula (L): 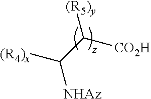
in the presence of a source of palladium (II) and an oxidant, whereby a compound of formula (2) is formed, wherein:
X is CH₂ or O;
n is an integer selected from 0 and 1;
o and m are integers independently selected from 0, 1, and 2, wherein the sum of o and m is not greater than 4;
x and y are integers independently selected from 0 and 1;
z is an integer selected from 0, 1, and 2;
R¹ is selected from H and C₁-C₆-alkyl;
each R² and R³ is independently selected from the group consisting of C₁-C₆-alkyl, C₁-C₆-alkoxy, halo, C₁-C₆-haloalkyl, and (C₆-C₁₀-aryl)(C₁-C₆-alkyl)-;
or an adjacent R² and R³, together with the carbon atoms to which they are bound, form a fused C₅-C₆-cycloalkyl or phenyl; and
each R⁴ and R⁵ is independently selected from the group consisting of H, C₁-C₆-alkyl, and (C₆-C₁₀-aryl)(C₁-C₆-alkyl)-;
or, when z is 1, then R⁴ and R⁵ together with the carbon atoms to which they are bound form a 5- to 6-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl.

2. The process according to claim 1, wherein X is CH₂.

3. The process according to claim 1, wherein X is O.

4. The process according to any one of claims 1 to 3, wherein n is 0.

5. The process according to any one of claims 1 to 3, wherein n is 1.

6. The process according to claim 1, wherein the compound of formula (2) is one selected from the following table:

| | |
|---|---|
| 2a | 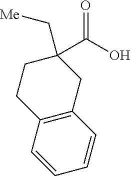 [SMILES: CCC1(C(=O)O)CCc2ccccc2C1] |
| 2b | 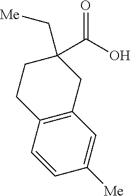 [SMILES: CCC1(C(=O)O)CCc2ccc(C)cc2C1] |
| 2c | 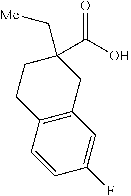 [SMILES: CCC1(C(=O)O)CCc2ccc(F)cc2C1] |
| 2d | 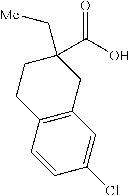 [SMILES: CCC1(C(=O)O)CCc2ccc(Cl)cc2C1] |
| 2e | 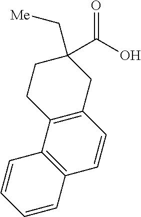 [SMILES: CCC1(C(=O)O)CCc2c(ccc3ccccc23)C1] |
| 2f | 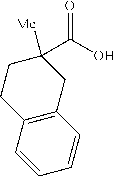 [SMILES: CC1(C(=O)O)CCc2ccccc2C1] |
| 2g | 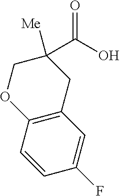 [SMILES: CC1(C(=O)O)COc2ccc(F)cc2C1] |
| 2h | 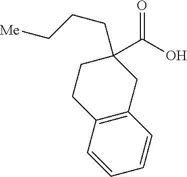 [SMILES: CCCCC1(C(=O)O)CCc2ccccc2C1] |
| 2i | 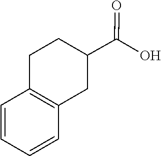 [SMILES: O=C(O)C1CCc2ccccc2C1] |
| 2j | 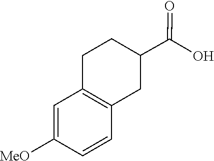 [SMILES: COc1ccc2c(c1)CCC(C(=O)O)C2] |
| 2k | 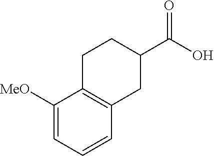 [SMILES: COc1cccc2c1CCC(C(=O)O)C2] |
| 2l | 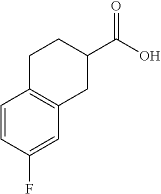 [SMILES: O=C(O)C1CCc2ccc(F)cc2C1] |
| 2m | 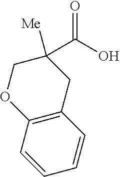 [SMILES: CC1(C(=O)O)COc2ccccc2C1] |
| 2n | 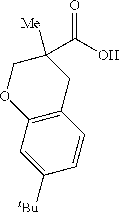 [SMILES: CC1(C(=O)O)COc2cc(C(C)(C)C)ccc2C1] |
| 2n′ | 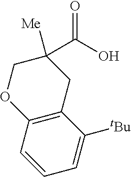 [SMILES: CC1(C(=O)O)COc2cccc(C(C)(C)C)c2C1] |
| 2o | 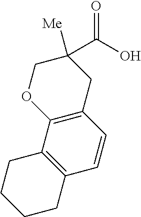 [SMILES: CC1(C(=O)O)COc2c(ccc3c2CCCC3)C1] |
| 2p | 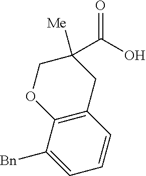 [SMILES: CC1(C(=O)O)COc2c(Cc3ccccc3)cccc2C1] |
| 2q | 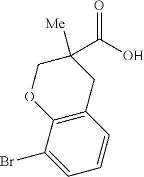 [SMILES: CC1(C(=O)O)COc2c(Br)cccc2C1] |
| 2r | 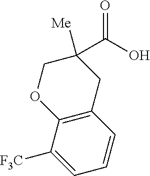 [SMILES: CC1(C(=O)O)COc2c(cccc2C(F)(F)F)C1] |
| 2s | 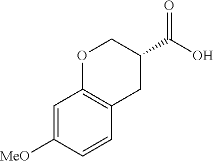 [SMILES: COc1ccc2c(c1)OC[C@H](C(=O)O)C2] |
| 2u | 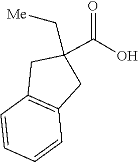 [SMILES: CCC1(C(=O)O)Cc2ccccc2C1] |
| 2v | 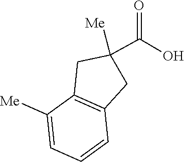 [SMILES: Cc1cccc2c1CC(C)(C(=O)O)C2] |
| 2w | 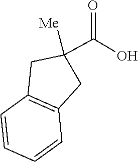 [SMILES: CC1(C(=O)O)Cc2ccccc2C1] |

7. The process according to any one of claims 1 to 6, wherein z is 1.

8. The process according to any one of claims 1 to 7, wherein one of x and y is 0 and the other is 1.

9. The process according to any one of claims 1 to 7, wherein R⁴ and R⁵ together with the carbon atoms to which they are bound form a 5- to 6-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl.

10. The process according to any one of claims 1 to 7 and 9, wherein R⁴ and R⁵ together with the carbon atoms to which they are bound form a 5-membered cycloalkyl, wherein the cycloalkyl group, in addition to having the —NHAc and the —CO₂H substituents as shown, is further optionally substituted with 1-2 substituents selected from the group consisting of halo, C₁-C₆-alkyl, C₁-C₆-alkoxy, and C₆-C₁₀-aryl.

11. The process according to any one of claims 1 to 7, wherein the ligand of formula (L) is one selected from the following table:

| | |
|---|---|
| L1 | 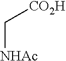 |
| L2 | 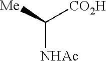 |
| L3 | 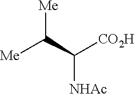 [SMILES: CC(=O)N[C@H](C(=O)O)C(C)C] |
| L4 | 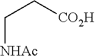 |
| L5 | 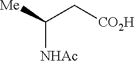 |
| L6 | 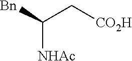 [SMILES: CC(=O)N[C@H](CC(=O)O)Cc1ccccc1] |
| L7 | 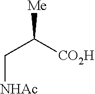 |
| L8 | 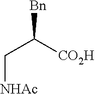 [SMILES: CC(=O)NC[C@@H](Cc1ccccc1)C(=O)O] |
| L9 | 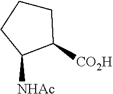 [SMILES: CC(=O)N[C@H]1CCC[C@H]1C(=O)O] |
| L10 | 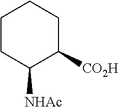 [SMILES: CC(=O)N[C@H]1CCCC[C@H]1C(=O)O] |
| L11 | 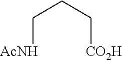 |

12. The process according to claim 11, wherein the ligand of formula (L) is L9:

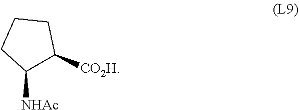 [SMILES: CC(=O)N[C@H]1CCC[C@H]1C(=O)O]

13. The process according to any one of claims 1 to 12, wherein the ligand of formula (L) is present in an amount of about 1 to about 15 mol % based upon the amount of compound of formula (2).

14. The process according to any one of claims 1 to 13, wherein the ligand of formula (L) is present in an amount of about 7 to about 12 mol %.

15. The process according to any one of claims 1 to 14, wherein the ligand of formula (L) is present in an amount of about 10 mol %.

16. The process according to any one of claims 1 to 15, wherein the source of palladium (II) is selected from Pd(OAc)₂ and Pd(CH₃CN)₄(BF₄)₂.

17. The process according to any one of claims 1 to 16, wherein the source of palladium (II) is present in amount of about 1 to about 15 mol % based upon the amount of compound of formula (2).

18. The process according to any one of claims 1 to 17, wherein the source of palladium (II) is present in amount of about 7 to about 12 mol %.

19. The process according to any one of claims 1 to 18, wherein the source of palladium (II) is present in amount of about 10 mol %.

20. The process according to any one of claims 1 to 19, wherein the oxidant is sodium percarbonate.

21. The process according to any one of claims 1 to 20, further comprising the contacting in the presence of LiOAc.

22. The process according to any one of claims 1 to 21, further comprising the contacting in the presence of hexafluoroisopropanol.

23. The process according to claim 1, wherein
the ligand of formula (L) is (L9) present in an amount of about 10 mol %: 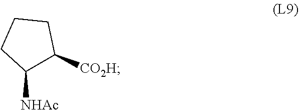 [SMILES: CC(=O)N[C@H]1CCC[C@H]1C(=O)O]
the sum of o and m is 1 or 2;
the source of palladium (II) is Pd(OAc)₂ in amount of about 10 mol %; and
the oxidant is sodium percarbonate.
